(19) United States
(12) Patent Application Publication
(10) Pub. No.: US 2026/0108622 A1
(43) Pub. Date: Apr. 23, 2026

(54) NON-PEGYLATED LIPID NANOPARTICLES

(71) Applicant: Massachusetts Institute of Technology, Cambridge, MA (US)

(72) Inventors: Paula T. Hammond-Cunningham, Newton, MA (US); Namita Rajesh Nabar, Cambridge, MA (US); Deeksha Kumaresh, Cambridge, MA (US)

(73) Assignee: Massachusetts Institute of Technology, Cambridge, MA (US)

(21) Appl. No.: 19/364,970

(22) Filed: Oct. 21, 2025

Related U.S. Application Data
(60) Provisional application No. 63/710,215, filed on Oct. 22, 2024.
(60) Provisional application No. 63/794,054, filed on Apr. 24, 2025.

(51) Int. Cl. A61K 47/69; A61K 9/00; A61K 31/7105; A61K 47/58; A61K 47/61; A61K 47/62; A61K 48/00; C12N 15/88

(52) U.S. Cl. CPC A61K 47/6911; A61K 9/0019; A61K 31/7105; A61K 47/58; A61K 47/61; A61K 47/62; A61K 48/0033; A61K 48/0083; C12N 15/88

(57) ABSTRACT

Provided herein are non-PEGylated lipid nanoparticles comprising an outer polyanionic layer, compositions thereof, and methods of using said particles or compositions for therapeutic applications.

### GOVERNMENT SUPPORT

This invention was made with government support under DGE2141064 awarded by the National Science Foundation. The government has certain rights in the invention.

### BACKGROUND

Lipid nanoparticles (LNPs) are biocompatible nanocarriers, certain of which have been shown to enable safe and effective non-viral delivery of nucleic acids. FDA-approved LNP therapies include Alnylam's Onpattro in 2018 (siRNA LNPs for hepatic transthyretin-mediated amyloidosis delivered intravenously) and Moderna and BioNTech/Pfizer's SARS-CoV-2 vaccines in 2020 (mRNA LNPs delivered intramuscularly), with multiple other LNP formulations in clinical trials for applications including cancers and vaccines.¹

LNPs can be stabilized by one of their component lipids, e.g., a polyethylene glycol (PEG)-lipid that protrudes from NP surfaces. PEG conjugated to a lipid anchor can reduce NP aggregation, and can decrease NP size and adsorption of other proteins. In in vivo applications, the steric hindrance provided by these lipids can reduce the surface adsorption of blood proteins (opsonization),2 thereby potentially delaying NP detection and clearance macrophages and increasing circulation times. However, recent administration of PEGylated LNPs for the COVID-19 vaccine revealed that PEG lipids can lead to the production of anti-PEG antibodies^{3,4}, causing lower circulation times especially with multiple doses (Accelerated Blood Clearance).^{5,6} In fact, in some cases, PEG lipids have been linked to a more severe reaction called complement activation-related pseudoallergy (CARPA), decreasing the safety of PEGylated nanocarriers.

### SUMMARY

In one aspect of the present disclosure, provided herein is a non-PEGylated nanoparticle comprising:
(a) a lipid nanoparticle (LNP) core comprising an ionizable or cationic lipid and a nucleic acid cargo; and
(b) an outer polyanionic layer, wherein:
   the LNP core does not comprise a PEG.

In another aspect, provided is a pharmaceutical composition comprising a plurality of nanoparticles of the present disclosure and a pharmaceutically acceptable excipient.

In another aspect, provided is a method of delivering a polynucleotide to a subject or cell, the method comprising administering to the subject or contacting the cell with a nanoparticle or the pharmaceutical composition described herein.

In another aspect, provided is a method of modulating transfection of a polynucleotide in a cell, the method comprising contacting the cell with the nanoparticle described herein.

In another aspect, provided is a method of treating or preventing disease in a subject in need thereof, the method comprising administering to the subject the nanoparticle or the pharmaceutical composition described herein.

In another aspect, provided herein is a method of stabilizing a non-PEGylated LNP, the method comprising electrostatic layering the non-PEGylated LNP with a polyanion, wherein the nanoparticle does not comprise a nucleic acid-based layer.

In another aspect, provided is use of a nanoparticle, or a pharmaceutical composition described herein, for the manufacture of a medicament for treating disease or disorder in a subject.

In another aspect, provided herein is a kit comprising:
a nanoparticle, or a pharmaceutical composition described herein; and
   instructions for using the particle or the pharmaceutical composition.

### BRIEF DESCRIPTION OF THE DRAWINGS

The accompanying drawings, which constitute a part of this specification, illustrate several embodiments of the invention and together with the description, provide non-limiting examples of the invention.

FIG. 1 is a schematic of the polyanion layering of non-PEGylated lipid nanoparticles (LNPs) disclosed herein.

FIGS. 2A-2B show physical characteristics of ALC non-PEGylated (nP) and PEGylated LNPs after formulation and after layering with poly (L-glutamic acid) (PLE), polyacrylic acid (PAA), poly-L-aspartic acid (PLD), and hyaluronic acid (HA). FIG. 2A shows nP LNPs were overall 20-50 nm larger in size than their PEG counterparts. FIG. 2B Charge reversal indicates complete layering and nP LLNPs were around 10-30 mV more negative than their PEG counterparts.

FIG. 3 shows the size of unlayered non-PEGylated (UL nonPEG) cKK LNPs versus PLE and PAA non-PEGylated LLNPs incubated in phosphate buffered saline (PBS) at room temperature for 28 h. N=3 technical replicates.

FIG. 4 shows the calculated size of non-PEGylated (nP) and PEGylated (PEG) LNPs and LLNPs incubated in plasma over time.

FIG. 5 shows negative staining room-temperature TEM images of UL (top) and PAA-layered (bottom) non-PEGylated LNPs upon 2 hour incubation in 50% mouse plasma at 37° C.

FIG. 6 shows in vitro transfection of non-PEGylated (nP) and PEGylated LNPs and LLNPs.

FIGS. 7A-7F show non-PEGylated PLE-LLNPs reduce liver transfection by ten-fold 4h after dosing. FIG. 7A shows an experimental schematic of C57BL6 mice dosed intravenously with PEGylated or non-PEGylated (nonPEG), unlayered (UL) or PLE-layered LNPs, containing luciferase-encoding mRNA, at 0.3 mg/kg. 4 h after dosing, clearance organs (spleen, liver, lungs, heart, kidneys) were harvested and evaluated for luciferase transfection. FIG. 7B shows ex vivo luciferase transfection of the spleen; luciferase signal is normalized by mass of tissue. FIG. 7C shows ex vivo luciferase transfection of the liver; luciferase signal is normalized by mass of tissue. FIG. 7D shows ex vivo luciferase transfection of the lungs; luciferase signal is normalized by mass of tissue. FIG. 7E shows ratio of weight-normalized spleen and liver transfection. FIG. 7F shows a table of physicochemical characterization (encapsulation efficiency (EE), diameter, polydispersity index (PDI), and zeta potential (ZP) of all NPs. N=3/group.

FIGS. 8A-8B show non-PEGylated LLNPs significantly improve transfection in macrophages. FIG. 8A shows transfection efficacy (% GFP) of RAW 264.7 macrophages after 4 hours incubation with PEGylated and non-PEGylated LNPs, either unlayered (UL) or layered with any of HA, PLE, or PAA, containing GFP-expressing mRNA. FIG. 8B shows transfection efficacy (% GFP) of RAW 264.7 macrophages after 24 hours incubation LNPs. N=3 biological replicates.

FIGS. 9A-9B shows non-PEGylated LNPs can be stably layered with a library of polyanions. FIG. 9A shows diameter (Z-average) and polydispersity index (PDI) of PEGylated and non-PEGylated LNPs layered with hyaluronic acid (HA), poly-L-glutamate (PLE), and polyacrylate (PAA). FIG. 9B shows surface zeta potential of PEGylated and non-PEGylated LNPs determined with dynamic light scattering in water. N=3 technical replicates.

FIGS. 10A-10B shows choice of outer layer on non-PEGylated LLNPs can confer NP stability in salt or plasma. FIG. 10A shows a table of physicochemical properties of non-PEGylated LNPs, either unlayered (UL), or coated with either PLE or PAA, in comparison to unlayered PEGylated control. FIG. 10B shows diameters of all LNP groups over time upon incubation in 50% C57BL6 mouse plasma at 37° C. N=3 technical replicates.

FIGS. 11A-11C show non-PEGylated layering approach is translatable across LNP cores varying in component lipids. FIG. 11A shows diameter (Z-average) and PDI (top) and surface zeta potential (bottom) of PEGylated or non-PEGylated, unlayered (UL) or layered, LNPs, with a core containing ALC-0315, FDA-approved lipid used in Pfizer-BioNTech COVID-19 vaccine. FIG. 11B shows diameter (Z-average) and PDI (top) and surface zeta potential (bottom) of PEGylated or non-PEGylated, unlayered (UL) or layered, LNPs, with a core containing SM-102, FDA-approved lipid used in Moderna COVID-19 vaccine. FIG. 11C shows diameter (Z-average) and PDI (top) and surface zeta potential (bottom) of PEGylated or non-PEGylated, unlayered (UL) or layered, LNPs, with a core containing cKK-E12, synthetic ionizable lipid. Physicochemical properties were determined with dynamic light scattering. N=3 technical replicates.

FIGS. 12A-12C shows PAA layer restores salt stability across cores. FIG. 12A shows the change in size over time of particles containing the ionizable lipid ALC-0315 incubated in phosphate buffered saline (PBS) at room temperature for 28 h. FIG. 12B shows the change in size over time of particles containing the ionizable lipid SM-102 incubated in phosphate buffered saline (PBS) at room temperature for 28 h. FIG. 12C shows the change in size over time of particles containing the ionizable lipid cKK-E12 incubated in phosphate buffered saline (PBS) at room temperature for 28 h. N=3 technical replicates. Control PEGylated unlayered LNPs using each ionizable lipid were also evaluated.

FIGS. 13A-13D show non-PEGylated (nP) LLNPs achieve improved or comparable transfection to PEG counterparts. FIG. 13A shows EGFP mean fluorescence intensity (MFI) for HEK293T epithelial cells dosed with PEGylated or non-PEGylated (nP) unlayered or layered LNPs, containing either ALC-0315 or cKK-E12, encapsulating GFP-encoding mRNA. FIG. 13B shows % EGFP+RAW 264.7 macrophages dosed with PEGylated or non-PEGylated unlayered (UL) or layered LNPs, containing either ALC-0315 or cKK-E12 encapsulating GFP-encoding mRNA. FIG. 13C shows EGFP MFI for OVCAR8 human ovarian cancer cells dosed with PEGylated or non-PEGylated (nP) unlayered (UL) or layered LNPs, containing either ALC-0315 or cKK-E121 encapsulating GFP-encoding mRNA. FIG. 13D shows % EGFP+COV362 human ovarian cancer cells dosed with PEGylated or non-PEGylated (nP) unlayered (UL) or layered LNPs, containing either ALC-0315, cKK-E121, or SM-102 encapsulating GFP-encoding mRNA. Yellow boxes highlight the layered formulations, with meaningful transfection, where the non-PEG LLNPs demonstrated higher transfection than their PEGylated counterparts. In all cases, cells were evaluated 24 h after dosing via flow cytometry. HEK293T and OVCAR8 transfection is reported as median fluorescence intensity (MFI), since all NP groups achieved >90% transfection. N=3-9 biological replicates.

FIGS. 14A-14F show non-PEGylated PLE-LLNPs reduce liver transfection by ten-fold 24 h after dosing. FIG. 14A shows an experimental schematic of C57BL6 mice dosed intravenously with PEGylated or non-PEGylated (noPEG), unlayered (UL) or PLE-layered LNPs, containing luciferase-encoding mRNA, at 0.3 mg/kg. 24 h after dosing, clearance organs (spleen, liver, lungs, heart, kidneys) were harvested and evaluated for luciferase transfection. FIG. 14B shows ex vivo luciferase transfection of the spleen; luciferase signal is normalized by mass of tissue. FIG. 14C shows ex vivo luciferase transfection of the liver; luciferase signal is normalized by mass of tissue. FIG. 14D shows ex vivo luciferase transfection of the lungs; luciferase signal is normalized by mass of tissue. FIG. 14E shows the ratio of weight-normalized spleen and liver transfection. FIG. 14F shows a table of physicochemical characterization (encapsulation efficiency (EE), diameter, polydispersity index (PDI), and zeta potential (ZP) of all NPs. N=3/group. Ordinary one-way ANOVA, Tukey's test post hoc. **p<0.01, *p<0.05, ns=not significant.

FIGS. 15A-15C show non-PEGylated PLE-LLNPs mitigate functional attenuation associated with accelerated blood clearance. FIG. 15A shows an experimental schematic C57BL6 mice dosed intravenously with PEGylated or non-PEGylated (nonPEG), unlayered (UL) or PLE-layered LNPs, containing luciferase-encoding mRNA, at 0.3 mg/kg. Mice were dosed once/week for three weeks. 24 h after each dose, live IVIS was conducted to quantify luciferase signal. FIG. 15B shows whole-body luminescence 24 h after each dose, in the case of PEGylated (left) and non-PEGylated (right) LLNPs. FIG. 15C shows quantification of attenuation of luciferase signal upon each successive dose, normalized to signal after Dose 1. N=5/group. Ordinary one-way ANOVA, Tukey's test post hoc. **p<0.01, *p<0.05, ns=not significant.

FIGS. 16A-16B show non-PEGylated PLE-LLNPs exhibit comparable circulation times to PEGylated LNPs. FIG. 16A shows an experimental schematic of C57BL6 mice dosed intravenously with PEGylated or non-PEGylated, unlayered or PLE-layered dye-labeled LNPs at 0.3 mg/kg. NP signal in sera from whole blood samples was quantified over 2 days. FIG. 16B shows normalized dye fluorescence detected in circulation over 50 hours, for each group. N=3/group.

### DEFINITIONS

The term “particle” refers to a small object, fragment, or piece of a substance that may be a single element, inorganic material, organic material, or mixture thereof. Examples of particles include polymeric particles, single-emulsion particles, double-emulsion particles, coacervates, liposomes, microparticles, nanoparticles (e.g., lipid nanoparticles), macroscopic particles, pellets, crystals, aggregates, composites, pulverized, milled or otherwise disrupted matrices, and cross-linked protein or polysaccharide particles. A particle may be composed of a single substance or multiple substances. In certain embodiments, the particle is a viral particle. In other embodiments, the particle is a liposome. In certain embodiments, the particle is a micelle. In certain embodiments, the particle is not a viral particle. In certain embodiments, the particle is not a micelle. In certain embodiments, the particle is substantially solid throughout. In certain embodiments, the particle is a nanoparticle. In certain embodiments, the particle is a microparticle. As used herein particle may refer to nanoparticle or lipid nanoparticle or LLNP.

The term “nanoparticle” refers to a particle having an average (e.g., mean) dimension (e.g., diameter) of between about 1 nanometer (nm) and about 1 micrometer (μm) (e.g., between about 1 nm and about 400 nm, about 1 nm and about 300 nm, between about 1 nm and about 100 nm, between about 1 nm and about 30 nm, between about 1 nm and about 10 nm, or between about 1 nm and about 3 nm), inclusive. Nanoparticles described herein were constructed using methods previously described by Correa, S., et al. ACS Nano 13, 5623-5634 (2019). As used herein in some embodiments, the terms “particle” and “nanoparticle” are interchangeable.

The term “polyelectrolyte” as used herein, refers to a polymer which under a particular set of conditions (e.g., physiological conditions) has a net positive or negative charge. In some embodiments, the polyelectrolyte is or comprises a polycation. In some embodiments, a polyelectrolyte is or comprises a polyanion. Polycations have a net positive charge and polyanions have a net negative change. The net charge of a given polyelectrolyte may depend on the surrounding chemical conditions (e.g., pH). Exemplary polyelectrolytes for use in polymeric coatings in the method of this disclosure include but are not limited to: poly(L-arginine) (PLR), polyethylenimine (PEI), poly-L-glutamic acid (PLE), polyarginine, polyglutamic acid, polylysine, poly-L-lysine (PLL), poly(β-amino esters), polystyrene sulfonate (SPS), polyacrylic acid (PAA), hyaluronic acid (HA), poly-L-aspartate, poly-L-aspartic acid (PLD), polyaspartic acid, polyaspartic acid, polyaminoacid composites, block co-polymers of poly amino acids and poly-ethylene glycol, polyacrylic acid, dextran sulfate, heparin folate, heparin sulfate, D-amino acid polymers or L/D amino acid mixtures, nucleic acids, fucoidan, sulfated-β-cyclodextran, polyglutamic acid-block-polyethylene glycol, linear poly(ethylene imine) (LPEI), poly(diallyldimethyl ammonium chloride) (PDAC), polyallylamine hydrochloride (PAH), poly(L-lactide-co-L-lysine, polyserine ester, poly(4-hydroxy-L-proline ester), poly[α(4-aminobutyl)-L-glycolic acid], sodium polystyrene sulfonate, dextran sulfate (DXS),, alginate, and chondroitin sulfate.

The term “polymer” refers to a compound comprising two or more covalently connected repeating units. In certain embodiments, a polymer is naturally occurring. In certain embodiments, a polymer is synthetic (i.e., not naturally occurring).

The term “cation” or “cationic” used herein refer to a species which has a net positive charge. The term “anion” or “anionic” used herein refer to a species which has a net negative charge.

The term “sterol” refers to a subgroup of steroids also known as steroid alcohols, i.e., a steroid containing at least one hydroxyl group. Sterols are usually divided into two classes: (1) plant sterols also known as “phytosterols,” and (2) animal sterols also known as “zoosterols.” The term “sterol” includes, but is not limited to, cholesterol, sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, and all natural or synthesized forms and derivatives thereof, including isomers.

As used herein, the term “associated with” refers to a direct association between two molecules, due to, for example, covalent, electrostatic hydrophobic, and ionic and/or hydrogen-bond interactions.

As used herein, the term “affinity” refers to the equilibrium constant for the reversible binding of two agents (e.g., tag and a ligand, or a metal ion and a ligand) and is expressed as a dissociation constant (KD). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10 -fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of a ligand for unrelated peptides or compounds. Affinity of a ligand to its binding partner can be, for example, from about 100 nanomolar (nM) to about 0.1 nM, from about 100 nM to about 1 picomolar (pM), or from about 100nM to about 1 femtomolar (fM) or more. An “affinity ligand”is a ligand having affinity for a binding partner.

Herein, the term “linker” (also known as “linker molecules” or “cross-linkers” or “spacers”) are molecules which may be used to conjugate one atom to another in a composition. The majority of known linkers react with amine, carboxyl, and sulfhydryl groups. Linker molecules may be responsible for different properties of the composition. The length of the linker should be considered in light of molecular flexibility during the conjugation step, and the availability of the conjugated molecule for its target. Longer linkers may improve the biological activity of the compositions as well as the ease of preparation of them. The geometry of the linker may be used to orient a molecule for optimal reaction with a target. A linker with flexible geometry may allow the entire composition to conformationally adapt as it binds a target sequence. The nature of the linker may be controlled by the monomeric units along with the polymer, e.g., a block polymer in which there is a block of hydrophobic monomers interspersed with a block of hydrophilic monomers.

The chemistry of preparing and utilizing a wide variety of molecular linkers is well-known in the art and many premade linkers for us in conjugating molecules are commercially available from vendors such as Pierce Chemical Co., Roche Molecular Biochemicals, United States Biological, VectorLabs, and BroadPharm.

Exemplary linker molecules for use in the compositions of the invention include, but are not limited to: propargylamine, aminocaproic acid (ACA); polyglycine, and any other amino acid polymers, polymethylmethacrylate (PMMA), polypropylene glycol (PPG); homobifunctional reagents such as APG, AEDP, BASED, BMB, BMDB, BMH, BMOE, BM [PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS; heterobifunctional reagents such as ABH, AEDP, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, MBUS, M2C2H, MPBH, MSA, NHS-ASA, PDPH, PMPI, SADP, SAED SAND, SANPAH, SASD, SATP, SBAP, SFAD, SIA, SIAB, SMCC, SMPB, SMPH, SMPT, SPDP, Sulfo-GMCS, Sulfo-GMBS, Sulfo-HSAB, Sulfo-KMUS, Sulfo-LC-SPDP, Sulfo-MBS, residues). Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS; and trifunctional linkers such as Sulfo-SBED. In certain embodiments, the linker comprises maleimide, and/or dibenzocyclooctyne (DBCO).

Branched linkers may be prepared or used so that multiple moieties per linker are able to react. Such multiply reactive linkers allow the creation of multimeric binding sites.

As used herein “targeting moiety” refers to a small molecule, protein, or fragment thereof which allows for or enhances binding to a target/target cell.

A “target cell” refers to a cell in a subject (in vivo) or ex vivo to which a compound, particle, and/or composition of the present disclosure is delivered. A target cell may be an abnormal or unhealthy, which may need to be treated. A target cell may also be a normal or healthy but is under a higher-than-normal risk of becoming abnormal or unhealthy, which may need to be prevented. In certain embodiments, the target cell is a cancer cell. In certain embodiments, the target cell is an ovarian cancer cell (e.g., HM-1 cell). In certain embodiments, the target cell is a colon cancer cell. In certain embodiments, the target cell is a brain cancer cell. In certain embodiments, the target cell is a skin cancer cell. In certain embodiments, the target cell is a head and neck cancer cell. In certain embodiments, the target cell is a lung cancer cell.

The term “pKa,” as used herein, includes the negative decadic logarithm of the ionization constant (Ka) of an acid; equal to the pH value at which equal concentrations of the acid and conjugate base forms of a substance (often a buffer) are present.

The term “hydrophobic,” as used herein, refers to a compound that has an octanol/water partition coefficient (K_{ow}) greater than about 10 at about 23° C.

The term “hydrophilic,” as used herein, refers to a compound that has an octanol/water partition coefficient (Kow) less than about 10 at about 23° C.

As used here, the term “PEG-lipid” refers to a PEGylated lipid.

An “amino acid” refers to natural and unnatural D/L alpha-amino acids, as well as natural and unnatural beta-and gamma-amino acids. A “peptide” refers to two amino acids joined by a peptide bond. A “polypeptide” refers to three or more amino acids joined by peptide bonds. An “amino acid side chain” refers to the group(s) pended to the alpha carbon (if an alpha amino acid), alpha and beta carbon (if a beta amino acid), or the alpha, beta, and gamma carbon (if a gamma amino acid).

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the present disclosure is delivered. A target tissue may be an abnormal or unhealthy tissue, which may need to be treated. A target tissue may also be a normal or healthy tissue that is under a higher-than-normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound, particle, and/or composition of the present disclosure described herein, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “agent,” “therapeutic agent,” or “pharmaceutical agent” are used herein to refer to any substance, compound (e.g., molecule), supramolecular complex, material, or combination or mixture thereof. A compound may be any agent that can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments, an agent may be provided as part of a composition, which may contain, e.g., a counter-ion, aqueous or non-aqueous diluent or carrier, buffer, preservative, or other ingredient, in addition to the agent, in various embodiments. In some embodiments, an agent may be provided as a salt, ester, hydrate, or solvate. In some embodiments, an agent is cell-permeable, e.g., within the range of typical agents that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates [e.g., with water (i.e. hydrates) or common solvents] and/or may have different crystalline forms (e.g., polymorphs) or different tautomeric forms. Embodiments exhibiting such alternative protonation states, configurations, solvates, and forms are encompassed by the present disclosure where applicable.

The terms “condition,” “disease,”and “disorder” are used interchangeably.

An “effective amount” of a particle or plurality of particles described herein refers to an amount sufficient to elicit the desired biological response. An effective amount of a particle or plurality of particles described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a particle or plurality of particles described herein in a single dose. In certain embodiments, an effective amount is the combined amounts of a particle or plurality of particles described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a particle or plurality of particles for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a particle or plurality of particles per unit dosage form.

In certain embodiments, the particle or plurality of particles of the present disclosure are administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a particle or plurality of particles described herein is an amount sufficient to provide a therapeutic benefit in the treatment of a condition or to delay or minimize one or more symptoms associated with the condition. A therapeutically effective amount of a particle or plurality of particles means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a particle or plurality of particles described herein is an amount sufficient to prevent a condition, or one or more symptoms associated with the condition or prevent its recurrence. A prophylactically effective amount of a particle or plurality of particles means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The term “chemical handle” as used herein, refers to a functional group installed for further chemical modification. In some embodiments, the chemical handle comprised an azide, alkyne, amine, carboxylic acid, thiol, or other targetable moiety. In some embodiments, the chemical handle is maleimide. In some embodiments, the chemical handle comprises an alkyne or a cycloalkyne. In some embodiments, the chemical handle is reactive to click chemistry.

The phrase “hepatic uptake” or “hepatic accumulation” as used herein described the specificity of nanoparticles (i.e., lipid nanoparticles) for uptake and/or degradation by the liver. LNPs preferentially accumulate in the liver, due in part to adsorption of serum proteins, that act as natural ligands for receptors on hepatocytes. In some embodiments, provided herein is a method of reducing hepatic uptake of nanoparticles by coating them with anionic polymers.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these. In some embodiments, a protein can be tethered to a particle. Exemplary proteins include but are not limited to interleukin-γ, interleukin-15 super agonist, interleukin 18, interferon-α, interferon-β, interferon-γ, interleukin-2, anti-PD1 antibodies, anti-PDL1 antibodies, anti-CTLA4 antibodies, anti-TIM-3 antibodies, anti-LAG-3 antibodies, anti-NKG2A antibodies, anti-CD73 antibodies, anti-A2aR antibodies, anti-B7-H3 antibodies, anti-B7-H4 antibodies, single-chain interleukin-12, tumor necrosis factor alpha, interleukin-10, interleukin-8, TNF-related apoptosis-inducing ligand (TRAIL), FMS-like tyrosine kinase 3 ligand (FLT3LG).

A cytokine is a signaling protein regulating biological functions such as innate and acquired immunity, hematopoiesis, inflammation and repair, and proliferation through mostly extracellular signaling. Interleukin 12 (11-12 or IL-12) is a cytokine that is naturally produced by dendritic cells, macrophages, neutrophils, and human B-lymphoblastoid cells in response to antigenic stimulation. Single chain interleukin 12 (scIL-12) is a protein in which the subunits of heterodimeric IL-12 are covalently bonded together. For example, interleukin 12 can be monomerized by introduction of a peptide linker between the subunits of heterodimeric cytokine. scIL-12 may be a fusion protein. Exemplary cytokines include but are not limited to interleukin-12 (IL-12), interleukin-2 (IL-2), interferon-γ (IFN-γ), interferon-α (IFN-α), interferon-β (IFN-β), interleukin-15 (IL-15), interleukin-21 (IL-21), interleukin-7 (IL-7), interleukin-15 super agonist (IL-15SA), interleukin-18 (IL-18), tumor necrosis factor alpha (TNF-α), interleukin-10 (IL-10), interleukin-8 (IL-8), TNF-related apoptosis-inducing ligand (TRAIL), FMS-like tyrosine kinase 3 ligand (FLT3LG), and variants thereof.

As used herein, a “polymer” refers to a compound comprised of at least 3 (e.g., at least 10, 20, 30, 40, 50, 60, 70, 80, 90, 100, etc.) repeating covalently bound structural units. In certain embodiments, a polymer is naturally occurring. In certain embodiments, a polymer is synthetic (i.e., not naturally occurring). In certain embodiments, the term “polymer” is used interchangeably with “polyelectrolyte.”A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed. ; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k. a. Wilms'tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k. a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

As used herein, a “small molecule” refers to an organic molecule with a molecular weight of less than 800 g/mol (e.g., less than 700 g/mol, less than 600 g/mol, less than 500 g/mol, less than 400 g/mol, less than 300 g/mol, less than 200 g/mol, less than 100 g/mol, between 50 to 800 g/mol, inclusive, between 100 to 800 g/mol, inclusive, or between 100 to 500 g/mol, inclusive). In certain embodiments, the small organic molecule is a therapeutically active agent such as a drug (e.g., a small organic molecule approved by the U.S. Food and Drug Administration as provided in the Code of Federal Regulations (CFR)). The small organic molecule may also comprise a metal. In some embodiments, small molecules can be tethered to a particle. Exemplary small molecules which can be tethered to a particle include but are not limited to Oxaliplatin, Doxorubicin, Paclitaxel, Lurbinectedi, Mitomycin, Trabectedin, Lobenguane, Lutetium, Radium, Cisplatin, or Sorafenib.

The terms “nucleic acid” or “nucleic acid sequence,” “nucleic acid molecule,” “nucleic acid fragment” or “polynucleotide” may be used interchangeably with “gene,” “mRNA encoded by a gene,”and “cDNA”.

The terms “assess,” “determine,” “evaluate,” and “assay” are used interchangeably herein to refer to any form of detection or measurement, and include determining whether a substance, signal, disease, condition, etc., is present or not. The result of an assessment may be expressed in qualitative and/or quantitative terms. Assessing may be relative or absolute. “Assessing the presence of” includes determining the amount of something that is present or determining whether it is present or absent.

Unless otherwise required by context, singular terms shall include pluralities, and plural terms shall include the singular.

“Dispersity” (D) as used herein is a measure of the distribution of molecular mass in a given polymer sample and is calculated by dividing the weight average molar mass (M_{w}) by the number average molar mass (Mₙ). The dispersity of a given sample can have a value equal to or greater than 1. As the polymer chains approach uniform chain length, the dispersity approaches unity (1). The dispersity of a polymer can be modified, for example, using polymer fractionation (e.g., preparative SEC, Baker-Williams fractionation, continuous spin fractionation), or modifying the work-up procedure (e.g., by partially dissolving a polymer, an insoluble high molar mass fraction may be filtered off resulting in a large reduction in Mw and a small reduction in Mn, thus reducing polydispersity).

As used herein, the term “salt” or “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

The terms “composition”and “formulation” are used interchangeably.

As used herein, forced methods of administration include convection-enhanced delivery and focused ultrasound.

### DETAILED DESCRIPTION OF CERTAIN EMBODIMENTS

Without wishing to be bound by theory, the inventors postulate that, because free PEG is minimally immunogenic, the linkage between PEG and lipid may be responsible for the immune response.² While polymeric and polypeptoid alternatives^{7,8,9} to PEG show promise, they are still conjugated to lipid anchors to incorporate into LNPs.

It was unexpectedly discovered that certain challenges of PEG-lipid-induced immunogenicity can be addressed through the electrostatic deposition of negatively charged polymers onto the surface of non-PEGylated LNPs. As used herein, the phrase “non-PEGylated” means the exclusion of any molecule comprising PEG, free or covalent associated. The additional electrolyte surface layer confers salt and plasma stability to LNPs without lipid anchors; furthermore, it is highly modular and can be applied to LNPs varying in lipid compositions.

Additionally, considering the varying reactivity of different polymers, layering can allow for greater customization of the particle for more targeted delivery.

The present disclosure describes the development of a non-PEGylated layered lipid nanoparticle (non-PEGylated LLNP), that can be used to encapsulate various nucleic acid cargos, including: messenger RNA (mRNA), plasmid DNA (pDNA), small interfering RNA (siRNA), and small guide RNAs (sgRNA).

In one aspect, provided herein is a non-PEGylated nanoparticle comprising:
a) a lipid nanoparticle (LNP) core comprising an ionizable or cationic lipid and a nucleic acid cargo; and
b) an outer polyanionic layer, wherein:
   the LNP core does not comprise a poly(ethylene glycol) (PEG).

In some embodiments, the LNP core does not comprise poly(ethylene glycol) (PEG), or PEG-derivatives. In some embodiments, the LNP core does not comprise PEG-derivatives such as PEGylated lipids or PEGylated polymers.

In some embodiments, the nanoparticle does not comprise a nucleic acid-based layer.

In some embodiments, the non-PEGylated nanoparticle comprises:
a) a lipid nanoparticle (LNP) core comprising an ionizable or cationic lipid and a nucleic acid cargo; and
b) an outer polyanionic layer, wherein:
   the LNP core does not comprise a poly(ethylene glycol) (PEG); and the nanoparticle does not comprise a nucleic acid-based layer

In some embodiments, the anionic polymer is not DNA or RNA. In some embodiments, the anionic polymer is not mRNA. In some embodiments, the particle does not comprise a poly-cationic polymer layer between the core and the anionic polymer. In some embodiments, the particle does not comprise a poly-arginine polymer layer between the core and the anionic polymer. In some embodiments, the LNP core does not comprise a poly-arginine.

In certain embodiments, the outer polyanionic layer comprises hyaluronic acid (HA), poly-L-glutamate (PLE), poly-L-aspartate (PLD), polyacrylic acid (PAA), dextran sulfate, chondroitin sulfate, fucoidan, heparin sulfate, alginate, polysialic acid, carboxymethyl cellulose, methacrylate, sulfated polybeta cyclodextrin.

In some embodiments, the anionic polymer is 5-120 kDa in molecular weight. In some embodiments, the anionic polymer is 10-20 kDa in molecular weight. In some embodiments, the anionic polymer is about 10-20 kDa in molecular weight. In some embodiments, the anionic polymer is about 10 kDa in molecular weight. In some embodiments, the anionic polymer is about 15 kDa in molecular weight. In some embodiments, the anionic polymer is about 20 kDa in molecular weight.

In some embodiments, the polyanionic layer comprises a chemical handle for conjugation (e.g., N₃, azide, NHS, maleimide, DBCO). In some embodiments, the polyanion comprises a chemical handle for conjugation (e.g., N₃, azide, NHS, maleimide, DBCO).

In some embodiments, the outer polyanionic layer comprises an anionic polymer. In certain embodiments, the anionic polymer is carboxylated or sulfonated. In some embodiments, the anionic polymer comprises sulfonate, carboxylate, or phosphonate moieties. In certain embodiments, the anionic polymer is carboxylated. In certain embodiments, the anionic polymer is sulfonated. In certain embodiments, the anionic polymer is an oligosaccharide (i.e. glucan). In certain embodiments, the anionic polymer is a protein. In certain embodiments, the anionic polymer is a homoprotein. In certain embodiments, the anionic polymer is a peptide. In certain embodiments, the anionic polymer is a homopeptide. In certain embodiments, the anionic polymer is synthetic. In some embodiments, the anionic polymer comprises a chemical handle.

In certain embodiments, the outer polyanionic layer comprises hyaluronic acid (HA). In certain embodiments, the outer polyanionic layer comprises poly-L-aspartate (PLD). In certain embodiments, the outer polyanionic layer comprises dextran sulfate. In certain embodiments, the outer polyanionic layer comprises chondroitin sulfate. In certain embodiments, the outer polyanionic layer comprises fucoidan. In certain embodiments, the outer polyanionic layer comprises heparin sulfate. In certain embodiments, the outer polyanionic layer comprises alginate. In certain embodiments, the outer polyanionic layer comprises polysialic acid. In certain embodiments, the outer polyanionic layer comprises carboxymethyl cellulose. In certain embodiments, the outer polyanionic layer comprises methacrylate. In certain embodiments, the outer polyanionic layer comprises sulfated polybeta cyclodextrin. In certain embodiments, the outer polyanionic layer comprises a sulfonated polymer. In certain embodiments, the outer polyanionic layer comprises a macrophage-targeting polymer.

In some embodiments, the outer polyanionic layer comprises PLE.

In certain embodiments, the outer polyanionic layer comprises PAA.

In some embodiments, the ionizable or cationic lipid is selected from the group consisting of ALC-0315, DLin-MC3-DMA, DLin-KC2-DMA, cKK-E12, C12-200, SM-102, C14-4, 306-O12B, and DOTAP. In some embodiments, the ionizable or cationic lipid is ALC-0315. In some embodiments, the ionizable or cationic lipid is DLin-MC3-DMA. In some embodiments, the ionizable or cationic lipid is DLin-KC2-DMA. In some embodiments, the ionizable or cationic lipid is cKK-E12, C12-200. In some embodiments, the ionizable or cationic lipid is SM-102. In some embodiments, the ionizable or cationic lipid is C14-4. In some embodiments, the ionizable or cationic lipid is 306-O12B. In some embodiments, the ionizable or cationic lipid is DOTAP.

In some embodiments, the ionizable lipid is ALC-0315. In some embodiments, the ionizable lipid is DLin-MC3-DMA. In some embodiments, the ionizable lipid is DLin-KC2-DMA. In some embodiments, the ionizable lipid is cKK-E12, C12-200. In some embodiments, the ionizable lipid is SM-102. In some embodiments, the ionizable lipid is C14-4. In some embodiments, the ionizable lipid is 306-O12B. In some embodiments, the ionizable lipid is DOTAP.

In some embodiments, the cationic lipid is ALC-0315. In some embodiments, the cationic lipid is DLin-MC3-DMA. In some embodiments, the cationic lipid is DLin-KC2-DMA. In some embodiments, the cationic lipid is cKK-E12, C12-200. In some embodiments, the cationic lipid is SM-102. In some embodiments, the cationic lipid is C14-4. In some embodiments, the cationic lipid is 306-O12B. In some embodiments, the cationic lipid is DOTAP.

In some embodiments, the lipid is ionizable. In some embodiments, the lipid is cationic.

In certain embodiments, the ionizable lipid is selected from the group consisting of ALC-0315, cKK-E12, and SM-102.

In some embodiments, the core further comprises a phospholipid.

In certain embodiments, the phospholipid is selected from the group consisting of 1,2-dioleoyl-sn-glycero-3-phosphoethanolamine (DOPE), 1,2-distearoyl-sn-glycero-3-phosphocholine (DSPC), hydrogenated soy phosphatidylcholine (HSPC), 1,2-dipalmitoyl-sn-glycero-3-phosphocholine (DPPC), 1,2-distearoyl-sn-glycero-3-phosphoethanolamine (DSPE), 1,2-dioleoyl-sn-glycero-3-phosphoethanolamine (DOPE), 1,2-Dipalmitoyl-sn-glycero-3-phosphoethanolamine (DPPE), 1,2-dioleoyl-sn-glycero-3-phosphocholine (DOPC), 1,2-dioleoyl-sn-glycero-3-phosphoethanolamine-N-[4-(p-maleimidophenyl)butyramide] (MPB-PE), 1,2-dioleoyl-sn-glycero-3-phosphoethanolamine-maleimide (DOPE-Mal), 1,2-dioleoyl-sn-glycero-3-phosphoethanolamine-N-hydroxysuccinimide (DOPE-NHS), 1,2-dioleoyl-sn-glycero-3-phosphoethanolamine-azide (DOPE-azide), 1,2-dioleoyl-sn-glycero-3-phosphoethanolamine-dibenzocyclooctyne (DOPE-DBCO), and 1,2-dioleoyl-sn-glycero-3-phosphoethanolamine-trans-cyclooctene (DOPE-TCO).

In some embodiments, the phospholipid comprises a chemical handle for conjugation (e.g., N₃, azide, NHS, maleimide, DBCO). In certain embodiments, the phospholipid is DOPE. In certain embodiments, the phospholipid is DSPC. In certain embodiments, the phospholipid is HSPC. In certain embodiments, the phospholipid is DPPC. In certain embodiments, the phospholipid is DSPE. In certain embodiments, the phospholipid is DPPE. In certain embodiments, the phospholipid is DOPC. In certain embodiments, the phospholipid is MPB-PE. In certain embodiments, the phospholipid is DOPE-Mal. In certain embodiments, the phospholipid is DOPE-NHS. In certain embodiments, the phospholipid is DOPE-azide. In certain embodiments, the phospholipid is DOPE-DBCO. In certain embodiments, the phospholipid is DOPE-TCO. In certain embodiments, the phospholipid is DOPE-N₃.

In some embodiments, the phospholipid is 1,2-dioleoyl-sn-glycero-3-phosphoethanolamine (DOPE).

In certain embodiments, the core further comprises a sterol.

In some embodiments, the sterol is selected from the group consisting of campesterol, desmosterol, stigmasterol, lanosterol, cholesterol, and sitosterol.

In certain embodiments, the sterol is cholesterol. In some embodiments, the sterol is campesterol. In some embodiments, the sterol is desmosterol. In some embodiments, the sterol is stigmasterol. In some embodiments, the sterol is lanosterol. In some embodiments, the sterol is sitosterol.

In some embodiments, the nucleic acid cargo is a polynucleotide.

In some embodiments, the polynucleotide is DNA or RNA.

In some embodiments, the polynucleotide is DNA. In certain embodiments, the cargo is plasmid DNA (pDNA). In certain embodiments, the cargo is single-stranded DNA (ssDNA), double-stranded DNA (dsDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, or viral DNA.

In certain embodiments, the polynucleotide is RNA (mRNA), plasmid DNA (pDNA), small interfering RNA (siRNA), small guide RNA (sgRNA).

In certain embodiments, the polynucleotide is RNA. In certain embodiments, the cargo is RNA. In some embodiments, the RNA is coding RNA or non-coding RNA. In some embodiments, the coding RNA is messenger RNA (mRNA). In some embodiments, the RNA is precursor messenger RNA. In some embodiments, the non-coding RNA is double-stranded RNA, short hairpin RNA, microRNA, guide RNA, transfer RNA, antisense RNA, long non-coding RNA, signal recognition particle RNA, small cytoplasmic RNA, small nuclear RNA, ribosomal RNA, Piwi-interacting RNA, small nucleolar RNA, or spliced leader RNA. In some embodiments, the non-coding RNA is small interfering RNA. In some embodiments, the RNA is single-stranded RNA, heterogeneous nuclear RNA, satellite RNA, viral RNA, or viral satellite RNA. In some embodiments, the RNA is single guide RNA (sgRNA). In some embodiments, the RNA is prime editing guide RNA (pegRNA). In some embodiments, the RNA is a ribonucleoprotein (RNP) complex. In some embodiments, the RNP complex comprises Cas9 protein and sgRNA. In some embodiments, the agent is ribozyme or flexizyme. In some embodiments, the polynucleotide is a DNA. In some embodiments, the DNA is a plasmid DNA (pDNA).

In certain embodiments, the cargo is small interfering RNA (siRNA). In certain embodiments, the cargo is messenger RNA (mRNA). In certain embodiments, the cargo is single-stranded RNA (ssRNA), double-stranded RNA (dsRNA), small interfering RNA (siRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or IncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, or viral satellite RNA. In certain embodiments, the agent is an RNA that carries out RNA interference (RNAi). The phenomenon of RNAi is discussed in greater detail, for example, in the following references: Elbashir et al., 2001, Genes Dev., 15:188; Fire et al., 1998, Nature, 391:806; Tabara et al., b 1999, Cell, 99:123; Hammond et al., Nature, 2000, 404:293; Zamore et al., 2000, Cell, 101:25; Chakraborty, 2007, Curr. Drug Targets, 8:469; and Morris and Rossi, 2006, Gene Ther., 13:553. In certain embodiments, upon delivery of an RNA into a subject, tissue, or cell, the RNA is able to interfere with the expression of a specific gene in the subject, tissue, or cell. In certain embodiments, the agent is a pDNA, siRNA, mRNA, or a combination thereof.

In some embodiments, the cargo is a CRISPR/Cas9 cargo (e.g., Cas9 mRNA/single guide RNA).

In certain embodiments, the polynucleotide is selected from the group consisting of mRNA, siRNA, sgRNA, cDNA, and pDNA. In certain embodiments, the polynucleotide is selected from the group consisting of mRNA, siRNA, sgRNA, and pDNA.

In some embodiments, the polynucleotide is mRNA. In certain embodiments, the polynucleotide is siRNA. In certain embodiments, the polynucleotide is sgRNA. In certain embodiments, the polynucleotide is cDNA. In certain embodiments, the polynucleotide is pDNA. In some embodiments, the polynucleotide is self-amplifying mRNA (saRNA). In some embodiments, the polynucleotide is circular RNA.

In certain embodiments, nanoparticle further comprises at least one targeting moiety or immunostimulatory molecule. In certain embodiments, nanoparticle further comprises a targeting moiety. In certain embodiments, nanoparticle further comprises a immunostimulatory molecule.

In some embodiments, the targeting moiety or immunostimulatory molecule is a protein or peptide. In some embodiments, the targeting moiety is a protein or peptide. In some embodiments, the immunostimulatory molecule is a protein or peptide.

In some embodiments, the targeting moiety or immunostimulatory molecule is selected from the group consisting of an antibody, nanobody, affibody, aptamer, cyclodextrin, a cytokine, and a derivative or fragment thereof.

In some embodiments, the targeting moiety is selected from the group consisting of an antibody, nanobody, affibody, aptamer, cyclodextrin, a cytokine, and a derivative or fragment thereof. In some embodiments, the targeting moiety is a protein, peptide, saccharide, or nucleic acid. In certain embodiments, the targeting moiety is selected from an antibody, nanobody, affibody, aptamer, cyclodextrin, or a derivative or fragment thereof. In certain embodiments, the targeting moiety is an antibody. In certain embodiments, the targeting moiety is nanobody. In certain embodiments, the targeting moiety is affibody. In certain embodiments, the targeting moiety is aptamer. In certain embodiments, the targeting moiety is cyclodextrin. In certain embodiments, the targeting moiety is an scFv. In certain embodiments, the targeting moiety is a peptide. In some embodiments, the peptide is SV40 T antigen NLS, M9 NLS, AP2, or RAP12. In some embodiments, the targeting moiety is SV40 T antigen NLS. In some embodiments, the targeting moiety is M9 NLS. In some embodiments, the targeting moiety is AP2. In some embodiments, the targeting moiety is RAP12.

In some embodiments, the targeting moiety targets stem cells, immune cell, or cancer cells. In some embodiments, the targeting moiety targets healthy cells. In some embodiments, the targeting moiety targets immune cells. In some embodiments, the targeting moiety targets white blood cells, leukocytes, or B-cells. In some embodiments, the targeting moiety targets cancer cells. In certain embodiments, the targeting moiety targets stem cells. In some embodiments, the stem cells are hematopoietic progenitor stem cells (HSPC). In certain embodiments, the targeting moiety targets cancer cells. In some embodiments, the cancer cells are ovarian cancer cells or blood cancer cells. In certain embodiments, the targeting moiety targets ovarian tumor cells. In certain embodiments, the targeting moiety targets blood cancer cells. In certain embodiments, the targeting moiety targets sickle blood cells.

In some embodiments, the immunostimulatory molecule is selected from the group consisting of an antibody, nanobody, affibody, aptamer, cyclodextrin, a cytokine, and a derivative or fragment thereof. In some embodiments, the immunostimulatory molecule is a protein, peptide, saccharide, or nucleic acid. In certain embodiments, the immunostimulatory molecule is selected from an antibody, nanobody, affibody, aptamer, cyclodextrin, or a derivative or fragment thereof. In certain embodiments, the targeting moiety is an antibody. In certain embodiments, the immunostimulatory molecule is nanobody. In certain embodiments, the immunostimulatory molecule is affibody. In certain embodiments, the immunostimulatory molecule is aptamer. In certain embodiments, the targeting moiety is cyclodextrin. In certain embodiments, immunostimulatory molecule is an scFv.

In some embodiments, the targeting moiety or immunostimulatory molecule is conjugated to a layer of the LNP.

In certain embodiments, the targeting moiety is an antibody or nanobody.

In some embodiments, the antibody or nanobody is an anti-CD117, anti-CD105, anti-CD90, anti-CXCR4, anti-CD45, anti-CD4, anti-CD8, anti-CD3, anti-CD19, anti-CD20, anti-PD1, anti-PDL1, anti-CTLA4, anti-TIM-3, anti-LAG-3, anti-NKG2A, anti-CD73, anti-A2aR, anti-B7-H3, anti-B7-H4, or anti-ferritin antibody or nanobody.

In some embodiments, the antibody or nanobody is an anti-CD117 antibody. In some embodiments, the antibody or nanobody is an anti-CD105 antibody. In some embodiments, the antibody or nanobody is an anti-CD90 antibody In some embodiments, the antibody or nanobody is an anti-CXCR4 antibody. In some embodiments, the antibody or nanobody is an anti-CD45 antibody. In some embodiments, the antibody or nanobody is an anti-CD4 antibody. In some embodiments, the antibody or nanobody is an anti-CD8 antibody. In some embodiments, the antibody or nanobody is an anti-CD3 antibody. In some embodiments, the antibody or nanobody is an anti-CD19 antibody. In some embodiments, the antibody or nanobody is an anti-CD20 antibody. In some embodiments, the antibody or nanobody is an anti-PD1 antibody. In some embodiments, the antibody or nanobody is an anti-PDL1 antibody. In some embodiments, the antibody or nanobody is an anti-CTLA4 antibody. In some embodiments, the antibody or nanobody is an anti-TIM-3 antibody. In some embodiments, the antibody or nanobody is an anti-LAG-3 antibody. In some embodiments, the antibody or nanobody is an anti-NKG2A antibody. In some embodiments, the antibody or nanobody is an anti-CD73 antibody. In some embodiments, the antibody or nanobody is an anti-A2aR antibody. In some embodiments, the antibody or nanobody is an anti-B7H3 antibody. In some embodiments, the antibody or nanobody is an anti-B7H4 antibody. In some embodiments, the antibody or nanobody is an anti-anti-ferritin antibody.

In some embodiments, the antibody is a nanobody. In some embodiments, the nanobody is an anti-CD117 nanobody, anti-CD45 nanobody, anti-CD105 nanobody, anti-CD90 nanobody, anti-CXCR4 nanobody, anti-CD4 nanobody, anti-CD8 nanobody, anti-CD3 nanobody, anti-CD19 nanobody, anti-CD20 nanobody, or anti-ferritin nanobody.

In certain embodiments, the immunostimulatory agent is a cytokine.

In some embodiments, the cytokine is selected from the group consisting of interleukin-12 (IL-12), interleukin-2 (IL-2), interferon-γ (IFN-γ), interferon-α (IFN-α), interferon-β (IFN-β), interleukin-15 (IL-15), interleukin-21 (IL-21), interleukin-7 (IL-7), interleukin-15 super agonist (IL-15SA), interleukin-18 (IL-18), tumor necrosis factor alpha (TNF-α), interleukin-10 (IL-10), interleukin-8 (IL-8), TNF-related apoptosis-inducing ligand (TRAIL), FMS-like tyrosine kinase 3 ligand (FLT3LG), and variants thereof. In some embodiments, the cytokine is interleukin-12 (IL-12). In some embodiments, the cytokine is interleukin-2 (IL-2). In some embodiments, the cytokine is interferon-γ (IFN-γ). In some embodiments, the cytokine is interferon-a (IFN-a). In some embodiments, the cytokine is interferon-β (IFN-β). In some embodiments, the cytokine is interleukin-15 (IL-15). In some embodiments, the cytokine is interleukin-21 (IL-21). In some embodiments, the cytokine is interleukin-7 (IL-7). In some embodiments, the cytokine is interleukin-15 super agonist (IL-15SA). In some embodiments, the cytokine is interleukin-18 (IL-18). In some embodiments, the cytokine is tumor necrosis factor alpha (TNF-α). In some embodiments, the cytokine is interleukin-10 (IL-10). In some embodiments, the cytokine is interleukin-8 (IL-8). In some embodiments, the cytokine is TNF-related apoptosis-inducing ligand (TRAIL). In some embodiments, the cytokine is FMS-like tyrosine kinase 3 ligand (FLT3LG).

In certain embodiments, the targeting moiety or immunostimulatory molecule is covalently bound or electrostatically associated to the LNP. In certain embodiments, the targeting moiety or immunostimulatory molecule is covalently bound to the LNP core. In certain embodiments, the targeting moiety or immunostimulatory molecule is electrostatically associated with the LNP core.

In some embodiments, the targeting moiety or immunostimulatory molecule is covalently bound to the anionic polymer. In certain embodiments, the targeting moiety or immunostimulatory molecule is covalently bound to the anionic polymer by click chemistry. In certain embodiments, the targeting moiety or immunostimulatory molecule is covalently bound to the LNP core by click chemistry.

In certain embodiments, the linker comprises maleimide and dibenzocyclooctyne (DBCO). In certain embodiments, the linker comprises maleimide. In certain embodiments, the linker comprises DBCO.

In some embodiments, the targeting moiety is bound to the anionic polymer by a linker. In some embodiments, the linker comprises a chemical handle (i.e., clickable handle (azide, alkyne etc.)) In some embodiments, the targeting moiety or immunostimulatory molecule is covalently bound to PAA. In some embodiments, the targeting moiety or immunostimulatory molecule is covalently bound to PLE. In some embodiments, the targeting moiety or immunostimulatory molecule is covalently bound to HA. In some embodiments, the targeting moiety or immunostimulatory molecule is covalently bound to PLD.

In certain embodiments, the nanoparticle further comprises an additional therapeutic agent. In some embodiments, the additional pharmaceutical agent is selected from the group consisting of a chemotherapeutic, targeted therapy, gene therapy, immune therapy, and hormone therapy.

In certain embodiments:
a) the LNP core comprises:
   i. ALC-0315;
   ii. cholesterol;
   iii. DOPE; and
b) the nucleic acid cargo comprises mRNA; and
c) the outer polyanionic layer comprises PLE or PAA.

In some embodiments, the nanoparticle comprises: a positively charged lipid nanoparticle core comprising cholesterol, a phospholipid selected from DOPC, DSPC, and DOPE, and an ionizable lipid selected from ALC-0315, DLin-MC3-DMA, DLin-KC2-DMA, cKK-E12, C12-200, 306-O12B, SM-102, and DOTAP; wherein the LNP core is electrostatically coated with a the polyanion PAA, HA, PLD, PLE, dextran sulfate, chondroitin sulfate, fucoidan, heparin sulfate, alginate, polysialic acid, carboxymethyl cellulose, methacrylate, or sulfated polybeta cyclodextrin; and a nucleic acid cargo.

In certain embodiments, the LNP core comprises 45-55 mol % of the ionizable lipid or cationic lipid. In certain embodiments, the LNP core comprises about 45-55 mol % of the ionizable lipid or cationic lipid. In certain embodiments, the LNP core comprises 45-50 mol % of the ionizable lipid or cationic lipid. In certain embodiments, the LNP core comprises 50-55 mol % of the ionizable lipid or cationic lipid.

In some embodiments, the LNP core comprises about 45-55 mol % ALC-0315. In some embodiments, the LNP core comprises about 50 mol % ALC-0315. In some embodiments, the LNP core comprises 51 mol % ALC-0315.

In certain embodiments, the LNP core comprises about 35-45 mol % sterol. In certain embodiments, the LNP core comprises about 35-40 mol % cholesterol. In certain embodiments, the LNP core comprises about 40-45 mol % cholesterol. In certain embodiments, the LNP core comprises about 40 mol % cholesterol. In certain embodiments, the LNP core comprises 39 mol % cholesterol. In certain embodiments, the LNP core comprises about 35-45 mol % cholesterol.

In some embodiments, the LNP core comprises about 5-15 mol % of the phospholipid. In some embodiments, the LNP core comprises about 5-10 mol % of the phospholipid. In some embodiments, the LNP core comprises about 10-15 mol % of the phospholipid. In some embodiments, the LNP core comprises about 5-15 mol % DOPE. In some embodiments, the LNP core comprises about 10 mol % DOPE. In some embodiments, the LNP core comprises 10 mol % DOPE. In some embodiments, the LNP core comprises 51 mol % ALC-0315, 39 mol % cholesterol, and 10 mol % DOPE.

In certain embodiments, the nanoparticle is about 50-300 nm in size. In certain embodiments, the nanoparticle is 75-150 nm in size. In certain embodiments, the nanoparticle is 75-100 nm in size. In certain embodiments, the nanoparticle is 100-125 nm in size. In certain embodiments, the nanoparticle is 100-150 nm in size. In certain embodiments, the nanoparticle is about 80 nm in size. In certain embodiments, the nanoparticle is about 90 nm in size. In certain embodiments, the nanoparticle is about 100 nm in size. In certain embodiments, the nanoparticle is about 110 nm in size. In certain embodiments, the nanoparticle is about 120 nm in size. In certain embodiments, the nanoparticle is about 125 nm in size. In certain embodiments, the nanoparticle is about 130 nm in size. In certain embodiments, the nanoparticle is about 140 nm in size. In certain embodiments, the nanoparticle is about 150 nm in size.

In certain embodiments, the cargo is a nucleic acid. In certain embodiments, the cargo is a pharmaceutical agent (i.e., small molecule, protein, or peptide). In some embodiments, there is more than one cargo. In some embodiments, the cargo comprises a protein. In some embodiments, the cargo comprises a complex of guide RNA and ribonucleoprotein (RNP). In some embodiments, the cargo comprises a cationic nuclear localization peptide covalently or electrostatically bound to pDNA. In some embodiments, the cargo is inside the LNP core. In some embodiments, the cargo is not layered around the core.

The present disclosure provides a pharmaceutical composition comprising a plurality of nanoparticles described herein and a pharmaceutically acceptable excipient. In certain embodiments, the pharmaceutical composition described herein comprises a plurality of LNPs described herein, and a pharmaceutically acceptable excipient.

In some embodiments, pharmaceutical composition further comprises an additional pharmaceutical agent.

In certain embodiments, the particle or plurality of particles described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for reducing the risk of developing a disease (e.g., proliferative disease, hematological disease, immune disorder, neurological disease, painful condition, psychiatric disorder, or metabolic disorder) in a subject in need thereof.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present ex vivo.

Pharmaceutical compositions described herein can be prepared by any method known in the art of pharmaceutics. In general, such preparatory methods include bringing the particle or plurality of particles described herein (i.e., the “active ingredient”) into association with a carrier or excipient, and/or one or more other accessory ingredients, and then, if necessary and/or desirable, shaping, and/or packaging the product into a desired single-or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents or fillers, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents or fillers include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, starches (such as dry starch, cornstarch), sugars (such as powdered sugar), calcium trisulfate, carboxymethylcellulose calcium, dextrate, dextrin, dextrose, fructose, lactitol, lactose, magnesium carbonate, magnesium, maltitol, maltodextrin, maltose, sucrose, glucose, mannitol, silicic acid, xylitol, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary disintegrating agents or disintegrants include agar, algin, alginic acid, sodium alginate, silicates, sodium carbonate, calcium carbonate, carboxymethylcellulose, cellulose, clay, colloidal silicon dioxide, croscarmellose sodium, crospovidone, rubber, magnesium silicate, methylcellulose, potassium krillin, hydroxypropylcellulose (e.g., low substituted Hydroxypropylcellulose), crosslinked polyvinylpyrrolidone, hydroxypropylcellulose, and starch (e.g., sodium glycolate starch, potato or tapioca starch).

Exemplary binding agents include starch (e.g., glycolate starch, cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof.

Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include agar, ethyl oleate, ethyl laurate, glycerin, blyceryl palmitostearate, magnesium oxide, magnesium stearate, mannitol, poloxamer, glycol, sodium stearyl, sorbitol, zinc stearate, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono-or di-glycerides. In addition, fatty acids such as oleic acid are used in the preparation of injectables.

In some embodiments, injectable preparations of the compositions disclosed herein are in the form of a ready-to-use (“RTU”) preparation that can be directly administered to a subject. In some embodiments, the RTU preparation is a suspension. In some embodiments, the RTU preparation is a solution. In some embodiments, the RTU preparation is an emulsion. In some embodiments, injectable preparations of the compositions disclosed herein are in the form of a solid that is reconstituted prior to administration. In some embodiments, the solid is a lyophilized solid. In some embodiments, injectable preparations of the compositions disclosed herein are in the form of a liquid or suspension that is diluted prior to administration.

In some embodiments, the pharmaceutical compositions disclosed herein comprise a bulking agent. Bulking agents can be used, e.g., to improve the appearance of a solid composition, to provide visible “bulk” to demonstrate product quality or to facilitate preparation, e.g., of a solid composition prepared for reconstitution prior to administration. Bulking agents can be used for low dose (high potency) drugs that do not have the necessary bulk to support their own structure or provide a visible composition in a unit dosage form. Bulking agents are used in lyophilized formulations. Bulking agents provide a desirable structure for a lyophilized cake comprising pores that provide the means for vapor to escape from the product during lyophilization cycles, and facilitate dissolution on reconstitution. In some embodiments, the bulking agent is mannitol, lactose, sucrose, dextran, trehalose, povidone, dextran, glycine, isoleucine, methionine, or a cyclodextrin (e.g., (2-hydroxypropyl)-β-cyclodextrin).

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compounds, particles, and/or compositions of the present disclosure described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin.

Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension.

Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface-active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The particle or plurality of particles provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compound, particle, and/or composition of the present disclosure can be administered by any route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, a compound, particle, and/or composition/pharmaceutical composition described herein is suitable for topical administration to the eye of a subject.

The exact amount of a compound/particle required to achieve an effective amount will vary from subject to subject, depending, for example, on species, age, and general condition of a subject, severity of the side effects or disorder, identity of the particular compound/particle, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound/particle described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a particle described herein. In certain embodiments, a dose described herein includes independently between 1 mg and 3 mg, inclusive, of a particle described herein. In certain embodiments, a dose described herein includes independently between 3 mg and 10 mg, inclusive, of a particle described herein. In certain embodiments, a dose described herein includes independently between 10 mg and 30 mg, inclusive, of a particle described herein. In certain embodiments, a dose described herein includes independently between 30 mg and 100 mg, inclusive, of a particle described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

The particle or plurality of particles, as described herein, can be administered in combination with one or more additional pharmaceutical agents (e.g., therapeutically and/or prophylactically active agents). The particle or plurality of particles, or compositions, can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a particle or plurality of particles described herein and an additional pharmaceutical agent shows a synergistic effect that is absent in a pharmaceutical composition including one of the particle or plurality of particles and the additional pharmaceutical agent, but not both. In some embodiments, the additional pharmaceutical agent achieves a desired effect for the same disorder. In some embodiments, the additional pharmaceutical agent achieves different effects.

The particle or plurality of particles, or composition, can be administered concurrently with, prior to, or subsequent to one or more additional pharmaceutical agents, which may be useful as, e.g., combination therapies. Pharmaceutical agents include therapeutically active agents. As used herein, therapeutic agent and pharmaceutical agent are used interchangeably. Pharmaceutical agents also include prophylactically active agents.

Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound, particle, and/or composition of the present disclosure in a single dose or composition or administered separately in different doses or compositions. The particular combination to employ in a regimen will take into account compatibility of the particle described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the particle or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

In another aspect, provided is a method of delivering a polynucleotide to a subject or cell, the method comprising administering to the subject or contacting the cell with a nanoparticle or the pharmaceutical composition described herein.

In certain embodiments, the method further comprises reducing liver transfection relative to an unlayered nanoparticle.

In some embodiments, the method further comprises mitigating accelerated blood clearance of the nanoparticle relative to an unlayered nanoparticle.

In certain embodiments, the particle is administered via intravenous injection, intraperitoneal injection, intratumoral injection, intramuscular injection, intradermal injection, topically, trans-dermally, oral intake, inhalation, intrathecally, intranasally, buccal, intravesically, intra-arterially, or through forced methods. In certain embodiments, the particle is administered via intravenous injection. In certain embodiments, the particle is administered via intraperitoneal injection. In certain embodiments, the particle is administered via intratumoral injection. In certain embodiments, the particle is administered via intramuscular injection. In certain embodiments, the particle is administered via intradermal injection. In certain embodiments, the particle is administered topically. In certain embodiments, the particle is administered trans-dermally. In certain embodiments, the particle is administered via oral intake. In certain embodiments, the particle is administered via inhalation. In certain embodiments, the particle is administered intrathecally. In certain embodiments, the particle is administered intranasally. In certain embodiments, the particle is administered buccally. In certain embodiments, the particle is administered intravesically. In certain embodiments, the particle is administered intra-arterially. In certain embodiments, the particle is administered via forced methods.

In another aspect, provided is a method of modulating transfection of a polynucleotide in a cell, the method comprising contacting the cell with the nanoparticle described herein.

In some embodiments, the modulating is increasing.

In some embodiments the cell is an immune cell, a hematopoietic cell, a stem cell, or a cancer cell. In some embodiments, the target cell is an immune cell, a hematopoietic cell, a stem cell, or a cancer cell. In some embodiments, the cell is an immune cell (i.e., B-cell, lymphocyte, leukocyte, etc.). In some embodiments, the cell is a hematopoietic cell. In some embodiments, the cell is a stem cell. In some embodiments, the cell is a hematopoietic stem cell (i.e. HSPC). In some embodiments, the cell is a cancer cell. In some embodiments, the cell is an ovarian cancer cell. In some embodiments, the cell is a blood cancer cell. In some embodiments, the cell is a tumor cell. In some embodiments, the cell is a healthy cell. In some embodiments, the cell is not a diseased cell.

In certain embodiments, the cell is a CD117-presenting cell, CD105-presenting cell, CD90-presenting cell, CXCR4-presenting cell, CD45-presenting cell, CD4-presenting cell, CD8-presenting cell, CD3-presenting cell, CD19-presenting cell, CD20-presenting cell, or ferritin-presenting cell.

In certain embodiments, the cell is a CD117-presenting cell, CD105-presenting cell, CD90-presenting cell, CXCR4-presenting cell, CD45-presenting cell, CD4-presenting cell, CD8-presenting cell, CD3-presenting cell, CD19-presenting cell, CD20-presenting cell, or ferritin-presenting cell. In certain embodiments, the cell is a CD117-presenting cell. In certain embodiments, the cell is a CD105-presenting cell. In certain embodiments, the cell is a CD90-presenting cell. In certain embodiments, the cell is a CXCR4-presenting cell. In certain embodiments, the cell is a CD45-presenting cell. In certain embodiments, the cell is a CD4-presenting cell. In certain embodiments, the cell is a CD8-presenting cell. In certain embodiments, the cell is a CD3-presenting cell. In certain embodiments, the cell is a CD19-presenting cell. In certain embodiments, the cell is a CD20-presenting cell. In certain embodiments, the cell is a ferritin-presenting cell.

In certain embodiments, the cell is a cancer or tumor cell.

In some embodiments, the cancer cell is an ovarian cancer cell. In some embodiments, the cancer cell is a brain cancer cell. In some embodiments, the cancer cell is a blood cancer cell.

In certain embodiments, the cell is a macrophage.

In some embodiments, the modulating is decreasing.

In certain embodiments, the cell is a human cell.

In certain embodiments, the cell is a stem cell. In certain embodiments, the cell is a hematopoietic stem cell.

In some embodiments, the cell is a liver cell.

In certain embodiments, the cell is in vivo.

In some embodiments, the cell is in vitro.

In another aspect, provided is a method of treating or preventing disease in a subject in need thereof, the method comprising administering to the subject the nanoparticle or the pharmaceutical composition described herein.

In certain embodiments, the disease is a proliferative disease, an immune disorder, genetic disease, or a virus.

In some embodiments, the disease is a proliferative disease. In certain embodiments, the disease is an immune disorder. In certain embodiments, the disease is genetic disease. In certain embodiments, the disease is virus. In certain embodiments, the disease is an infection. In some embodiments, the proliferative disease is ovarian cancer, brain cancer, or blood cancer.

In another aspect, provided is a method of stabilizing a non-PEGylated LNP, the method comprising electrostatic layering the non-PEGylated LNP with a polyanion, wherein the nanoparticle does not comprise a nucleic acid-based layer.

In some embodiments, the polyanion is selected from the group consisting of hyaluronic acid (HA), polyglutamate (PLE), poly aspartic acid (PLD), and poly acrylic acid (PAA). In some embodiments, the polyanion is HA. In some embodiments, the polyanion is PLE. In some embodiments, the polyanion is PLD. In some embodiments, the polyanion is PAA. In some embodiments, the polyanion comprises HA. In some embodiments, the polyanion comprises PLE. In some embodiments, the polyanion comprises PLD. In some embodiments, the polyanion comprises PAA.

In another aspect, provided is use of a nanoparticle, or a pharmaceutical composition described herein, for the manufacture of a medicament for treating disease or disorder in a subject.

In certain embodiments, the nanoparticle or pharmaceutical composition described herein, is for use in treating a disease or disorder in a subject. In some embodiments, the disease or disorder is a proliferative disease. In certain embodiments, the disease or disorder is an immune disorder. In certain embodiments, the disease or disorder is genetic disease. In certain embodiments, the disease or disorder is virus. In certain embodiments, the disease or disorder is an infection.

In one aspect, provided is a method of editing a gene in a cell, the method comprising contacting the cell the nanoparticle or the pharmaceutical composition described herein.

In another aspect, provided herein is a kit comprising: a nanoparticle or a pharmaceutical composition described herein; and instructions for using the particle or the pharmaceutical composition.

The kits provided may comprise a pharmaceutical composition or particle or plurality of nanoparticles described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or particles described herein. In some embodiments, the pharmaceutical composition or particles described herein provided in the first container and the second container are combined to form one unit dosage form.

Thus, in one aspect, provided are kits including a first container comprising a nanoparticle or plurality of particles or pharmaceutical composition described herein. In certain embodiments, the kits are useful for treating a disease (e.g., proliferative disease, immune disease, or hematological disease) in a subject in need thereof. In certain embodiments, the kits are useful for preventing disease (e.g., proliferative disease, immune disease, or hematological disease) in a subject in need thereof. In certain embodiments, the kits are useful for reducing the risk of developing a disease (e.g., proliferative disease, immune disease, or hematological disease) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, immune disease, or hematological disease) in a subject in need thereof. In certain embodiments, the kits and instructions provide for preventing a disease (e.g., proliferative disease, immune disease, or hematological disease) in a subject in need thereof. In certain embodiments, the kits and instructions provide for reducing the risk of developing a disease (e.g., proliferative disease, immune disease, or hematological disease) in a subject in need thereof. A kit described herein may include one or more additional pharmaceutical agents described herein as a separate composition.

In some embodiments, the unlayered (UL) non-PEGylated LNP is formulated through mixing three component lipids: (1) an ionizable cationic lipid, (2) phospholipid, and (3) cholesterol or sterol derivative, and any of the previously listed nucleic acid cargos. For layering, the UL LNPs are kept positively charged in an aqueous buffer while added to a solution of negatively charged polymer under rapid spinning conditions.

### EXAMPLES

In order that the present disclosure may be more fully understood, the following examples are set forth. The synthetic and biological examples described in this application are offered to illustrate the particles, pharmaceutical compositions, and methods provided herein and are not to be construed in any way as limiting in their scope.

### Example 1: Formation and Stability

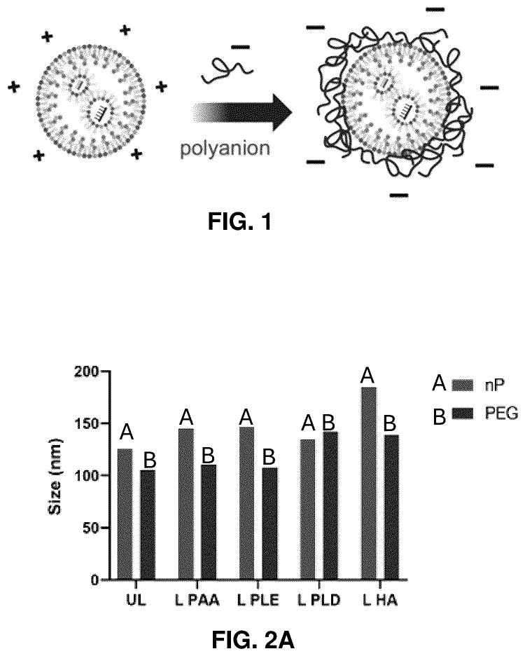

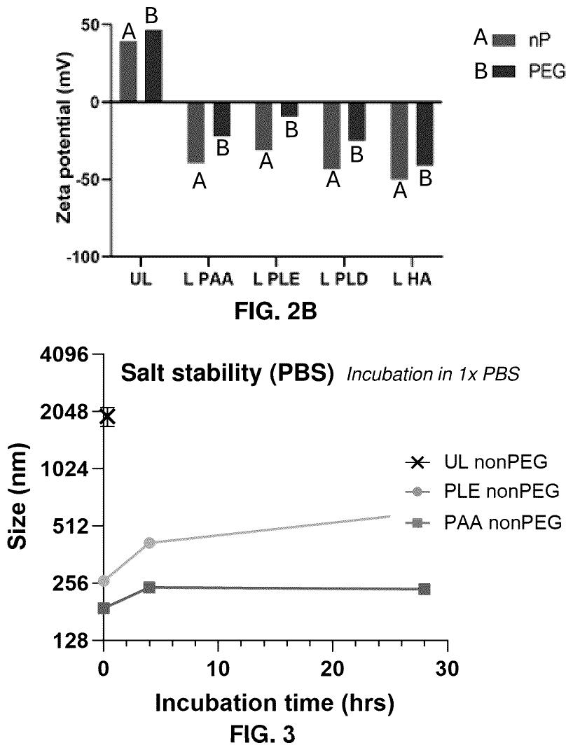

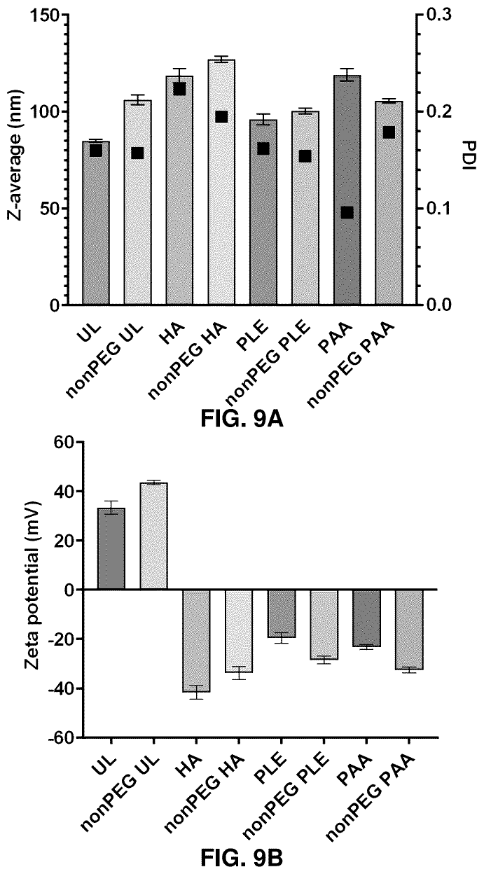

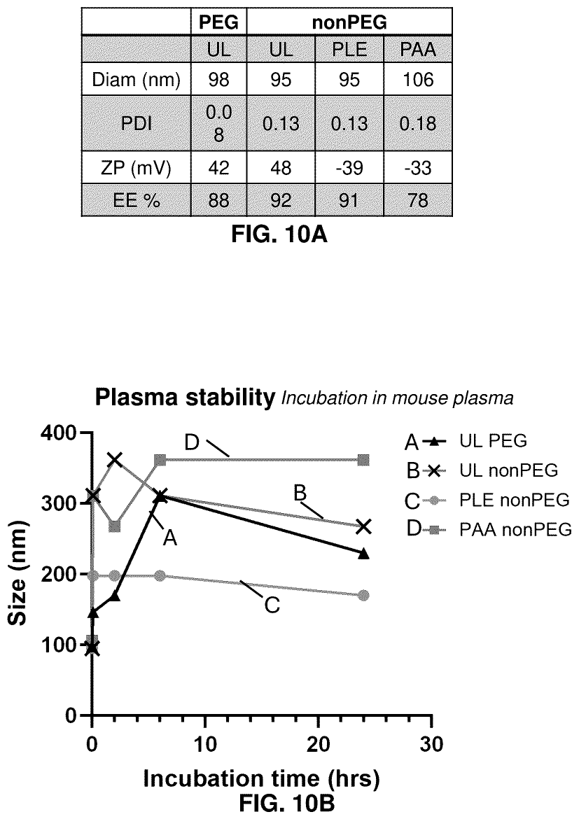

UL non-PEGylated LNPs can be formulated in water using gentle purification methods, such as dialysis. Non-PEGylated LNPs were generated and stably layered with each of a library of four carboxylated polyanions: hyaluronic acid (HA), poly-L-aspartate (PLD), poly-L-glutamate (PLE), and polyacrylate (PAA). Diameter, PDI, and surface zeta potential were determined with dynamic light scattering (DLS) in water. Stable layering is evidenced by LLNPs around ˜90-125 nm in size, diameter increases of ˜10-60 nm, and complete charge reversal, from cationic unlayered NPs to anionic layered NPs, +30 to −50 mV. Non-PEGylated layered LNPs formed with comparable size and zeta potential to PEGylated counterparts (FIGS. 1, 2A-2B, 9A-9B). After formulation of the LLNPs, excess polymer was removed through either dialysis or centrifugation.

Formation of UL LNPs in a saline solution is challenging, as is the standard for LNP formulation, because they may rapidly aggregate and crash out, evidenced by rapid growth in size after incubation in PBS and observed physical clouding of solution (FIG. 3). The salt stability of the non-PEGylated LLNPs was determined by taking size readings over a period time of incubation in PBS. Salt stability is particularly relevant to in vivo performance as blood contains many salts that can influence the size of the non-PEGylated LNPs, thereby affecting their performance. Unlike the UL non-PEGylated LNPs, the non-PEGylated LLNPs do not rapidly aggregate in saline solution, revealing the salt stability benefits of layering (FIG. 3).

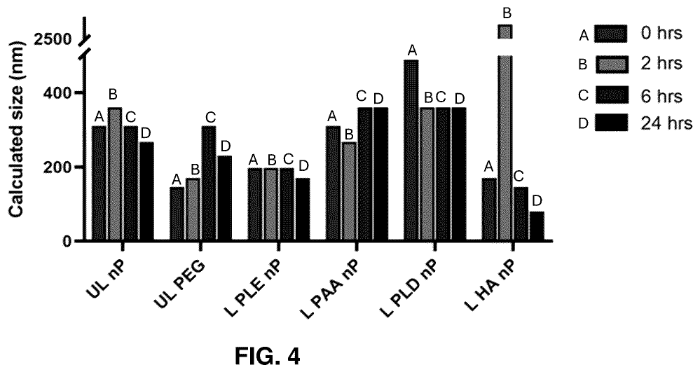

Next, the plasma stability of the non-PEGylated (nP) particles was tested to determine the effect of opsonization on the size of the particles. Specifically, degradation and/or aggregation of the particles without PEG support was of concern. Particles were incubated in 50% plasma solution and DLS measurements were taken at different time points. The DLS result was then deconvoluted to reveal the size of the particles over the incubation period (FIG. 4). Immediately after incubation, UL nP LNPs were around 300 nm whereas UL PEG LNPs were smaller at around 200 nm, indicating better plasma stability with the PEG lipid. PLE layered nP particles seem to be stable at around 200 nm over the 24-hour period, also indicating better stability (comparable to that of UL PEG LNPs) than the UL nP LNPs. Within the first 2 hours of incubation, the UL non-PEGylated LNPs grew more than the UL PEG LNPs, highlighting the role of PEG in promoting particle stability and decreasing opsonization. However, PLE layering seems to provide comparable plasma stability benefits to PEG as the PLE non-PEGylated LLNPs maintained a similar size to the initial UL PEG LNP reading (FIG. 4) over the incubation period.

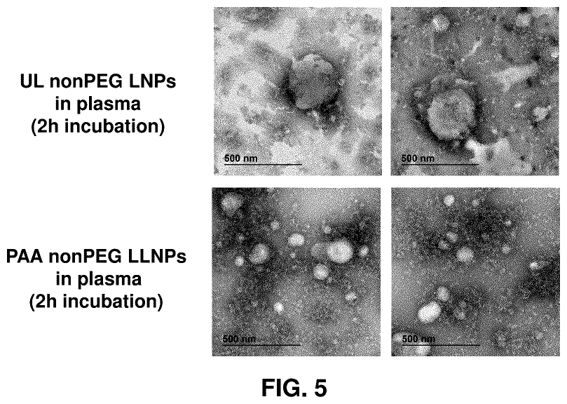

As shown in FIG. 5, TEM images of particles incubated in 10% plasma reveal UL non-PEGylated LNPs are intact, with sizes around 150-400 nm. Similarly, PAA layered non-PEGylated LNPs are intact, but with smaller sizes around 50-200 nm and a stable morphology in plasma, reiterating that layering confers plasma stability benefits. Also, the larger number of intact particles, considering that the initial concentration of both particles was the same, support the stability benefits of polyanion layering.

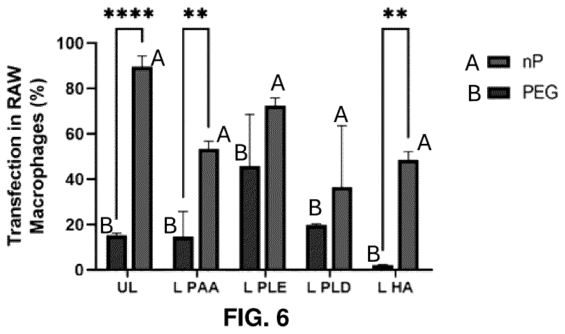

Next, the in vitro performance of the non-PEGylated LNPs and LLNPs was evaluated in hard-to-transfect RAW 264.7 macrophages. As shown in FIG. 6, nP LLNPs had higher transfection than the standard UL PEG LNP, with mean transfection efficiencies between 35-80%, relative to around 20% for UL PEG. nP LLNPs also overall had higher transfection than the PEGylated LLNPs: the transfection of PAA nP LLNP was around 50%, relative to that of PAA PEG LLNP around 17% and the transfection of HA nP LLNP was around 45%, relative to that of HA PEG LLNP around 5%.

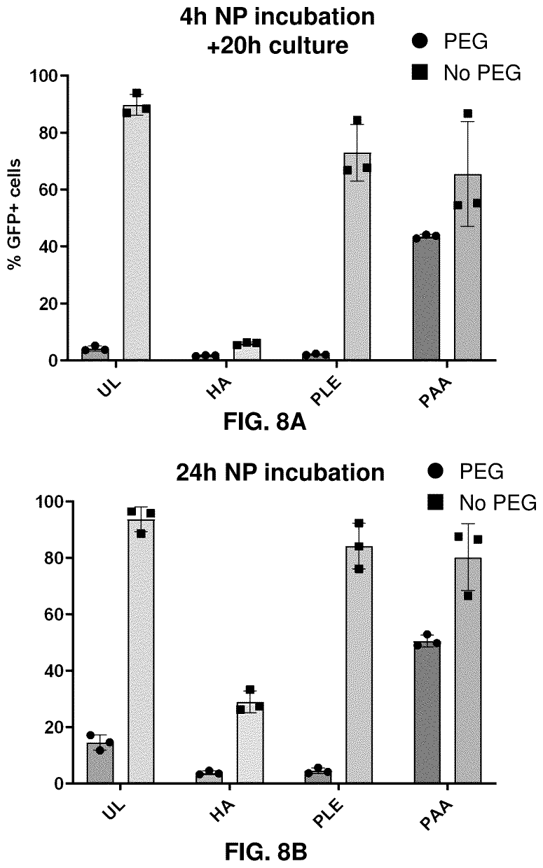

After NPs were incubated with cells for either 4h (FIG. 8A) or 24 h (FIG. 8B); transfection was evaluated 24 h after dosing via flow cytometry. Overall, the non-PEGylated LNPs and LLNPs had significantly improved transfection efficiencies than their PEGylated counterparts in RAW 264.7 macrophages.

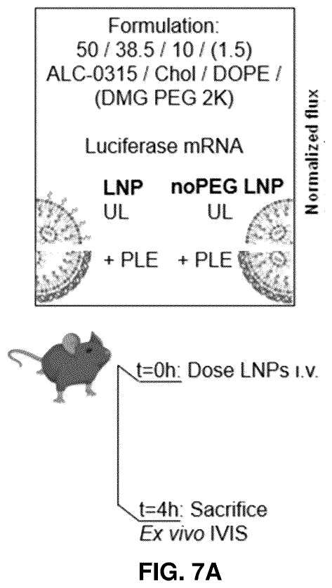

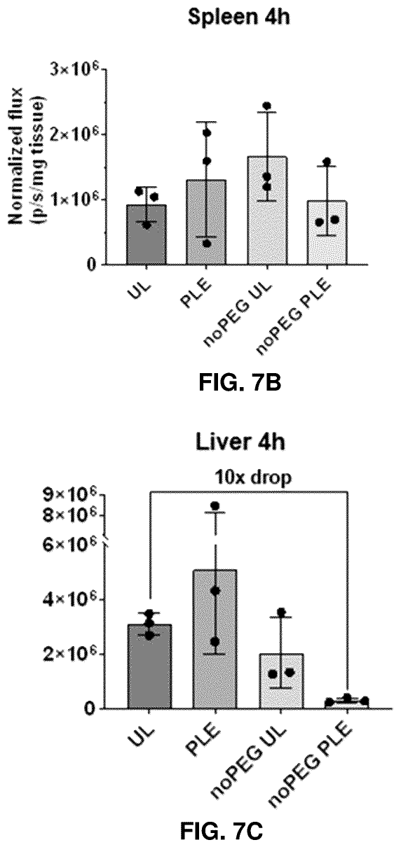

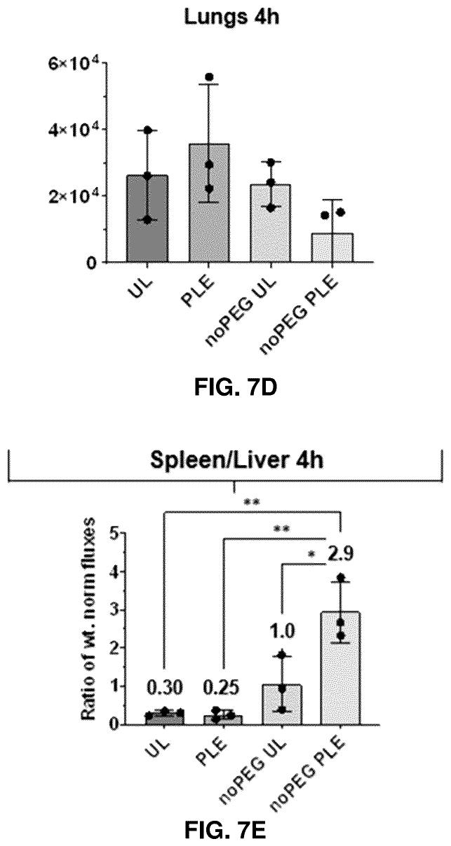

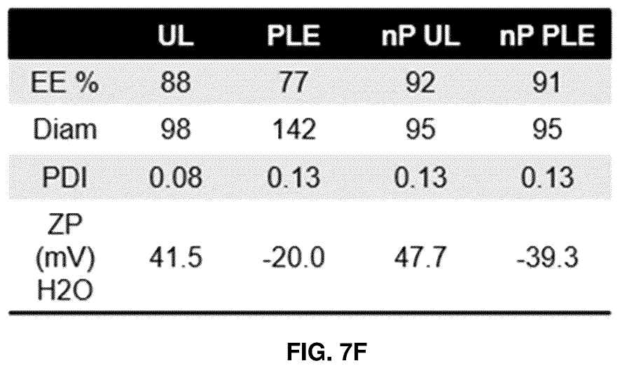

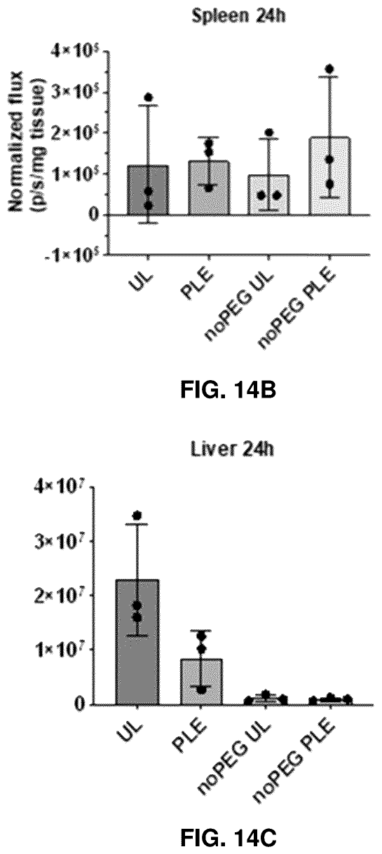

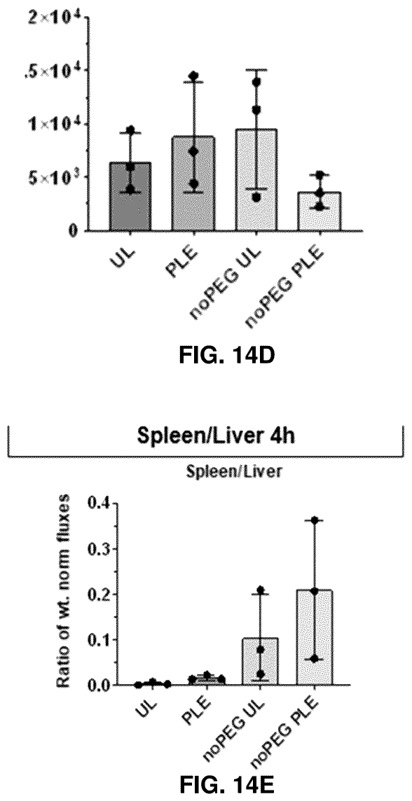

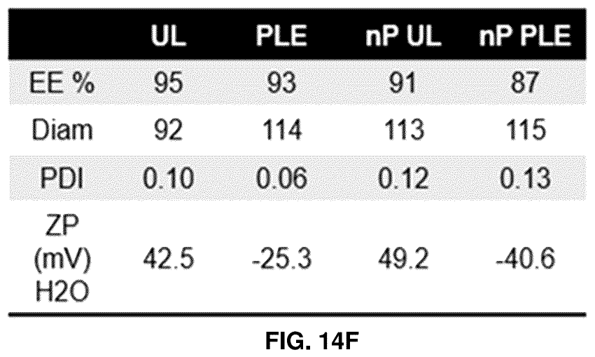

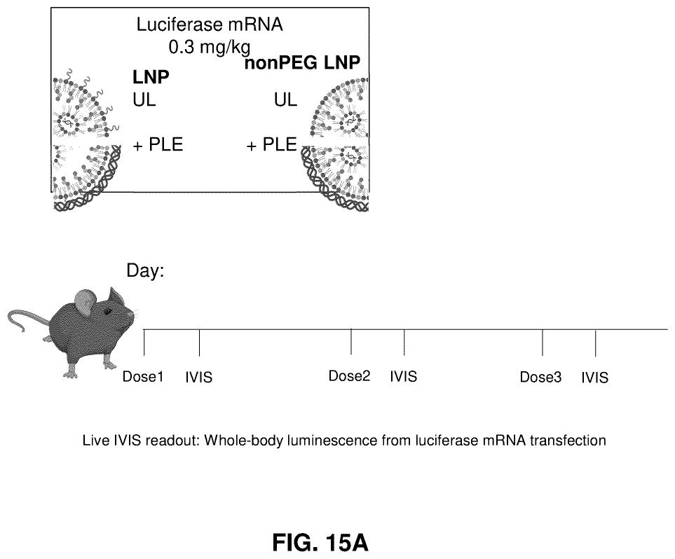

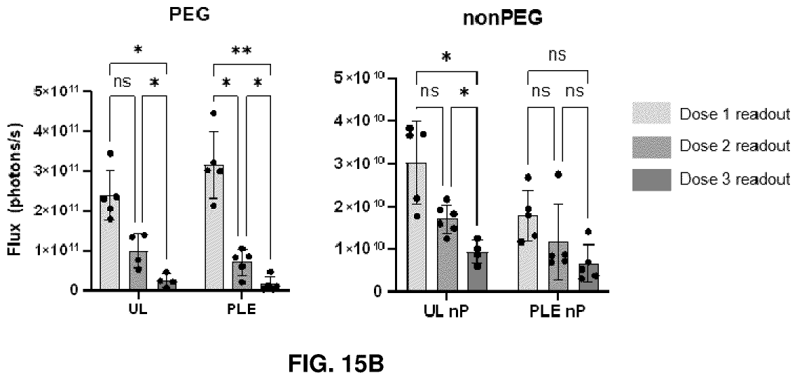

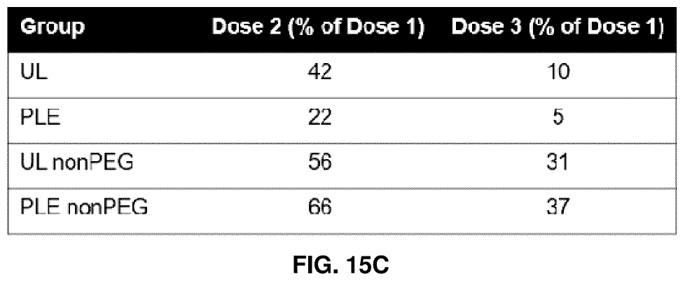

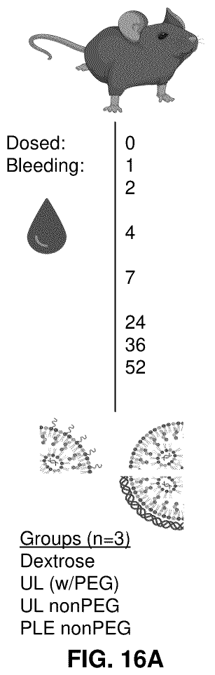

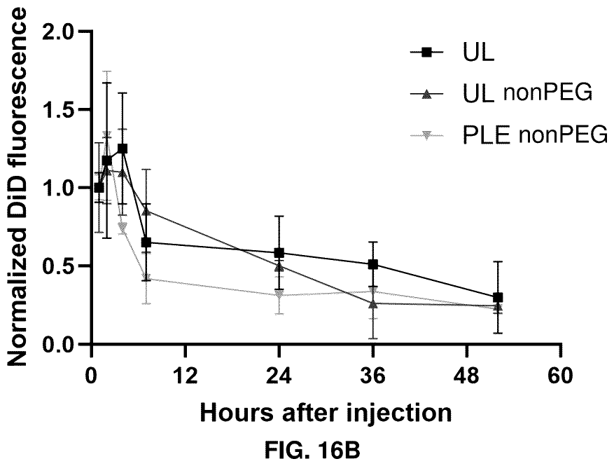

In vivo data further suggests the stability and transfection efficacy of non-PEGylated LLNPs. Healthy Black/6 mice were dosed with UL or PLE-LLNPs, either with or without PEG. While signal across many vascularized organs (spleen, lungs) remained consistent, non-PEGylated PLE-LLNPs were observed to reduce liver transfection by 10x after 4 and 24 hr, suggesting that the presence of the outer layer may help avoid hepatic accumulation and clearance (FIGS. 7, 14).

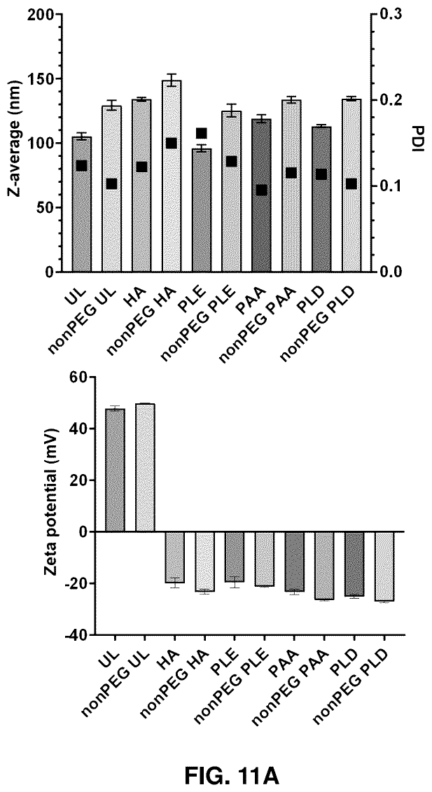

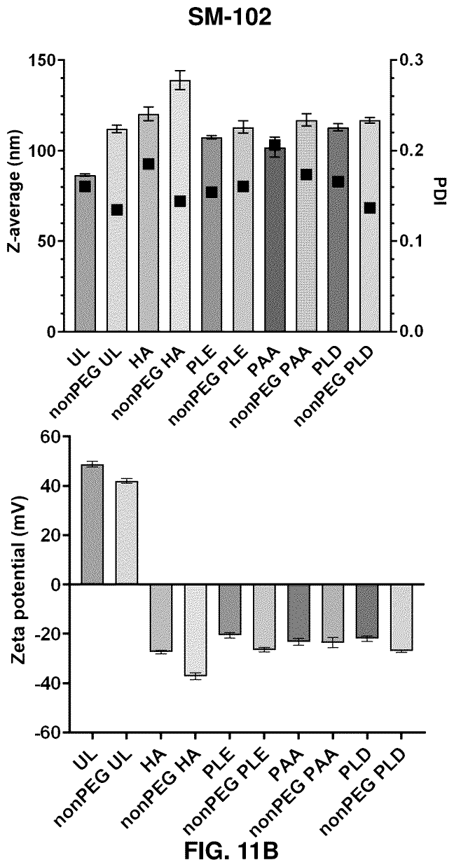

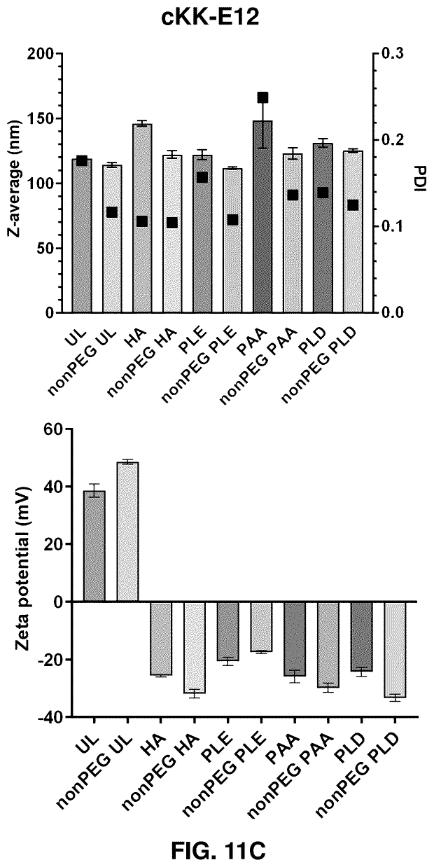

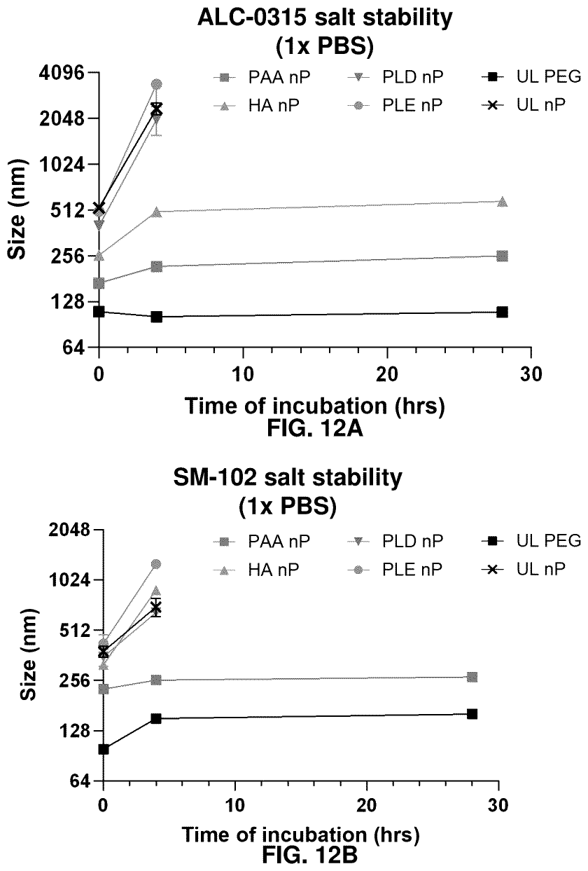

The non-PEGylated layering approach provided herein is translatable across LNP cores chemistries. FIGS. 11A-11C shows diameter, PDI, and surface zeta potential of PEGylated or non-PEGylated, unlayered or layered, LNPs, with cores containing ALC-0315 (FIG. 11A), FDA-approved lipid used in Pfizer-BioNTech COVID-19 vaccine, SM-102 (FIG. 11B), FDA-approved lipid used in Moderna COVID-19 vaccine, and cKK-E12 (FIG. 11C), synthetic ionizable lipid. Physicochemical properties were determined with dynamic light scattering. Stable layering is evidenced by diameter increases of 5-50 nm and complete charge reversal, from cationic unlayered NPs to anionic layered NPs. Non-PEGylated layered LNPs formed with comparable size and zeta potential to PEGylated counterparts.

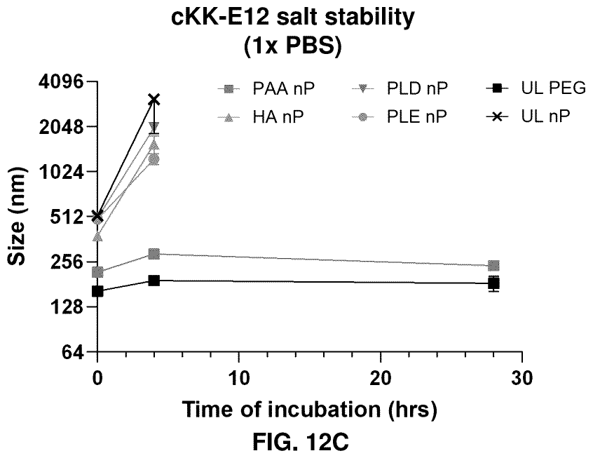

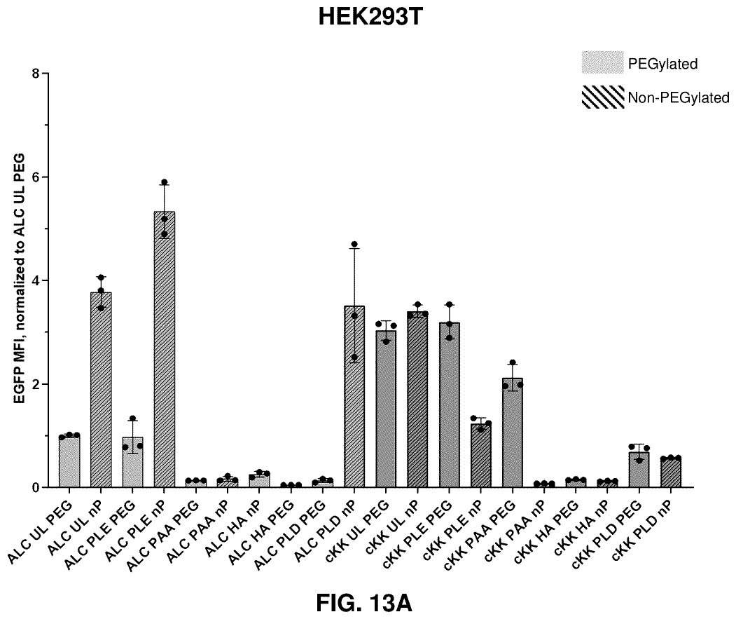

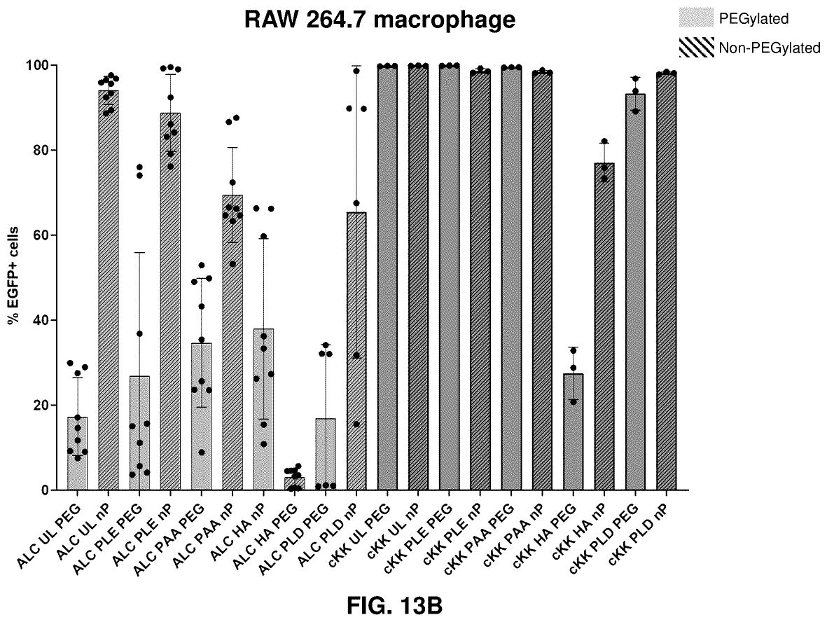

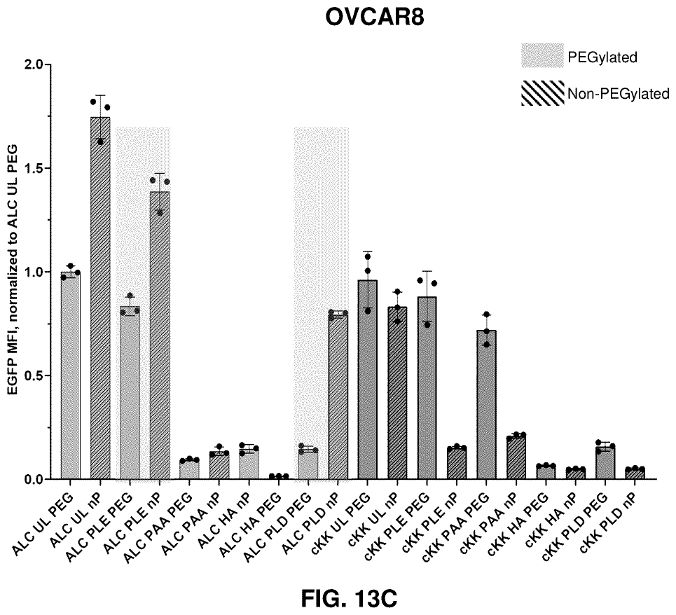

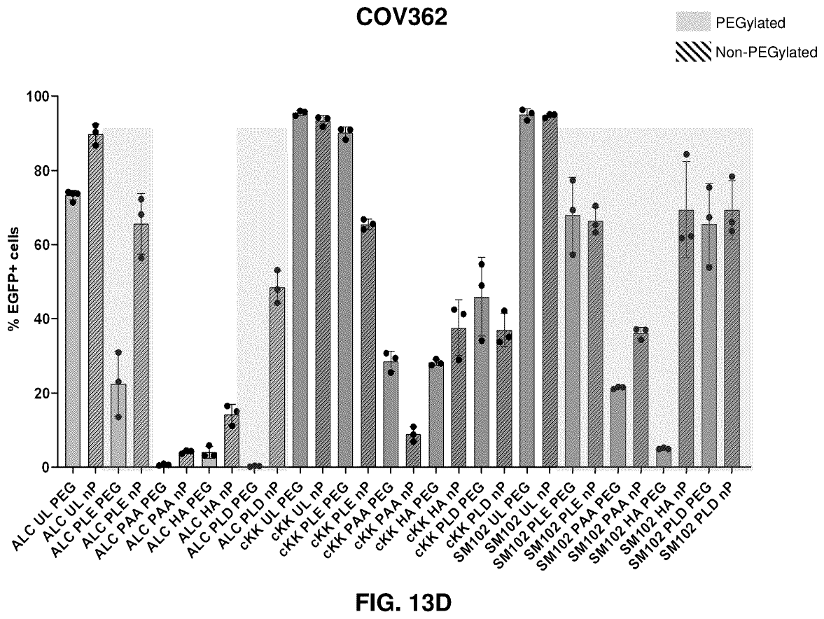

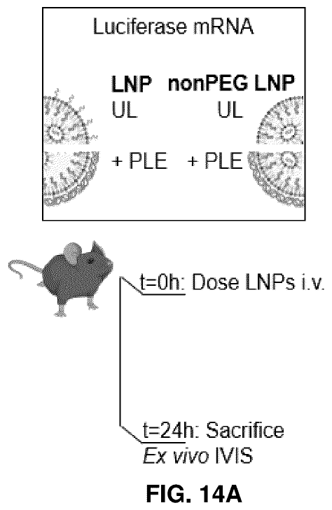

To assess stability in salt solution, these compositions were incubated in phosphate buffered saline (PBS) at room temperature for 28 h. It was determined that adsorbed surface layer of PAA restores salt stability on non-PEGylated LNPs across cores (FIGS. 12A-12C). Diameters were measured prior to incubation, immediately after incubation, and at 4 and 28 h, using dynamic light scattering. Control PEGylated unlayered LNPs using each ionizable lipid were also evaluated. LNPs that grew beyond 1000 nm were considered to have aggregated and were not further evaluated.

### Example 2: Materials and Methods

### Non-PEGylated LNP Synthesis

Non-PEGylated LNPs were formulated as described in US-2025-0082776-A1, the contents of which are incorporated herein by reference in their entirety. Briefly, component lipids: (DOPE, cholesterol, ALC-0315, SM-102, cKK-E12) were dissolved in 100% molecular biology-grade ethanol at concentrations ranging from 2.5 to 25 mg/ml, based on solubility limits. Lipids were co-dissolved in 100% ethanol at the appropriate molar ratios (51 mol % ionizable lipid, 39 mol % cholesterol, 10% phospholipid). mRNA was dissolved in 25 mM sodium acetate (pH 4.5) in an amber glass scintillation vial, with stir bars. To 4 volumes of nucleic acid, stirring at 700 rpm, 1 volume of lipid mixture was added in. The solution was allowed to rest for 5 minutes without stirring. Then, stirring at 700 rpm, the solution was diluted with 5 parts DNAse/RNAse-free water.¹⁰

Non-PEGylated LNPs were purified of ethanol and sodium acetate through dialysis against water, using Slide-a-Lyzers (3.5K MWCO) for 2 hours, changing dialysis buffer every 30 minutes. If further concentration was required, non-PEGylated LNPs were spun down in Amicon ultracentrifugal filter units, MWCO 100K, at 1000×g for 3-5 minutes. Prior to loading of NPs, Amicon units were primed with DNAse/RNAse free water. non-PEGylated LNPs were stored at 4° C.

### Non-pegylated LNP Layering

Polyanions tested included hyaluronic acid (HA), poly-L-glutamate (PLE), polyacrylate (PAA), and poly-L-aspartate (PLD). Polyanions were dissolved in DNAse/RNAse free water at 10 mg/ml, through vortexing and 10 minutes of sonication.

Stocks of HA, PLE, PAA, and PLD were diluted to 8, 2, 2, and 2 respective weight equivalents, relative to LNP lipid concentration, diluting in 5 mM HEPES. Polyanion baths were added to amber glass scintillation vials, with stir bars. Under stirring at 800 rpm, RT, an equal volume of purified unlayered LNPs in water was added. The mixture was stirred for 15 min, then incubated without stirring for 1 hour at RT. non-PEGylated LLNPs were purified of excess polyanion through washes in water in Amicon ultracentrifugal filter units, MWCO 100K, spinning at 2650 rpm, 3-5 minutes. Prior to loading of NPs, Amicon units were primed with DNAse/RNAse free water. non-PEGylated LLNPs were stored at 4° C.

### LNP Quantification and Encapsulation Efficiency

Encapsulation efficiency and concentration of purified unlayered and layered non-PEGylated LLNPs were determined using the Quant-it™ RiboGreen RNA assay kit, as previously published.¹⁰

### Salt Stability Assay

20 μL of non-PEGylated LLNPs were incubated in 1×PBS, RT, at a final concentration of 10 ng/μL. At specified timepoints, aliquots of the non-PEGylated LLNPs were diluted 8-fold in PBS; size and polydispersity index was measured using dynamic light scattering (DLS), on a Malvern Zetasizer.

### Plasma Stability Assay

20 μL of non-PEGylated LLNPs, at final concentrations ranging 5-30 ng/μL were mixed with 20 μL of C57BL6 mouse plasma (Innovative Research), and incubated shaking at 100 rpm, at 37° C., for up to 2 days. At specified timepoints, aliquots of the non-PEGylated LLNPs were diluted 50x into DNAse/RNAse free water; size and polydispersity index was measured using DLS, on a Malvern Zetasizer. Plasma without NPs was incubated under identical conditions and times, and measured on DLS as well. DLS data was post-processed by subtracting background signal of plasma-only condition.

### Negative-staining TEM

Unlayered or PAA-layered non-PEGylated LLNPs were incubated in 50% C57BL6 mouse plasma (Innovative Research) for 2 hours, shaking at 100 rpm, at 37° C. The LNPs and plasma were then diluted 10-fold in de-ionized water, and stained with uranyl acetate for imaging by the Koch Institute Nanotechnology core.

### In Vitro Yransfection Studies

Cell lines assessed include: HEK293T, RAW 264.7, OVCAR8, and COV362. 24 hours prior to dosing, cells were plated at 10,000 (HEK293T, RAW 264.7) or 20,000 (OVCAR8, COV362) per well. Higher plating densities were used for OVCAR8 and COV362 to ensure sufficient cell yield for flow cytometry. Cells were incubated at 37° C., 5% CO₂ overnight to ensure adherence. Next, cells were dosed with non-PEGylated or PEGylated unlayered or layered LNPs and incubated for an additional 24 hours. For flow cytometry preparation, cells were washed 1x in 100 uL PBS and dissociated with 5-10 minutes incubation with 30 uL 0.25% trypsin-EDTA. Trypsin-EDTA was neutralized with 150 uL complete media. Cells were washed 1x in PBS, then incubated for 10 minutes in a 1:500 dilution of Zombie Violet (viability dye) in PBS. Zombie Violet was neutralized with 1% BSA in PBS. Cells were washed 2x in 1% BSA in PBS, then read for GFP expression on either a BD FACSymphony A3 HTS or BD LSR Fortessa HTS.¹⁰

### In Vivo Transfection Studies

Female C57BL/6, 8-10 weeks of age, were acquired from Jackson Laboratories, and housed in MIT DCM facility. All animal experiments were approved by the MIT Committee on Animal Care (protocol #221000434).

### Biodistribution

Animals were dosed retro-orbitally with non-PEGylated or PEGylated unlayered or PLE-layered LNPs, containing firefly luciferase-encoding mRNA, at 0.3 mg/kg. At either 4 hours or 24 hours, animals were sacrificed, and key clearance organs (liver, spleen, lungs, heart, kidneys) were harvested and incubated in Roswell Park medium on ice. Organs were weighed, rinsed 1x in PBS, then soaked for 2 minutes in a solution of 1 mg/ml D-luciferin in PBS. Organs were then imaged on an IVIS machine for luminescence.

### Circulation

Animals were dosed retro-orbitally with non-PEGylated or PEGylated unlayered or PLE-layered LNPs, dyed with fluorophore DiD, at 0.3 mg/kg. Two cohorts of identically-dosed mice were used, to ensure individual mice were not excessively bled. At 1, 2, 4, 7, 24, 36, and 52 hours, blood was collected from dosed mice via retro-orbital bleed, in serum collection tubes. Tubes were rested at RT for 30 minutes, then spun at 10,000×g 5 minutes; serum was collected and read on a Tecan plate reader for DiD signal.

### Accelerated Blood Clearance

Once every 7 days, animals were dosed retro-orbitally with non-PEGylated or PEGylated unlayered or PLE-layered LNPs, containing firefly luciferase-encoding mRNA, at 0.3 mg/kg. 24 hours after dosing, animals were injected intraperitoneally with 200 uL of D-luciferin, and imaged live on IVIS for whole-body luminescence.¹⁰

### Conclusions

The particles of the present disclosure may display numerous advantages, including: lack of immunogenic PEG-conjugate, highly efficient intracellular nucleic acid delivery, greater salt and plasma stability, and greater extrahepatic nucleic acid delivery in vivo to increase targeted effects. Given that the inventors hypothesize that the linkage between PEG and lipid is the cause of immunogenicity, it is advantageous that this technology is an effective stabilizing agent without having to be conjugated to a lipid anchor.

### Example 3:

### Sex-Balanced Data

Biodistribution and transfection data of non-PEGylated LLNPs is collected at 4 hours, 24 hours in male mice, to have complete sex-balanced data set.

### PAA Non-PEGylated LLNPs

Another promising surface chemistry from stability studies is PAA. Transfection and circulation of non-PEGylated LLNPs layered with PAA is assessed using the same or similar protocols and assays described herein.

### Toxicity Profile To determine safety/toxicity profile of non-PEGylated LLNPs, CBC, liver enzymes, bilirubin, and related toxicity panels are measured.

### Immune Response

Comparison studies of immune response between PEGylated and non-PEGylated systems are competed using ELISA. The concentration of anti-PEG IgM, IgE, complement proteins and any cytokines associated with adverse reactions in the serum of dosed mice are assessed.

### Long-Term Storage To understand the feasibility of long-term storage, non-PEGylated LLNPs are lyophilized and stability overtime is assessed.

### REFERENCES

1. Thi, T. T. H., Suys, E. J. A., Lee, J. S., Nguyen, D. H., Park, K. D., & Truong, N .P. (2021). Lipid-Based Nanoparticles in the Clinic and Clinical Trials: From Cancer Nanomedicine to COVID-19 Vaccines. Vaccines, 9(4), 359. https://doi.org/10.3390/vaccines9040359
2. Tenchov, R., Sasso, J. M., & Zhou, Q. A. (2023). Bioconjugate chemistry. Bioconjugate Chemistry, 34(6), 941-960. https://doi.org/10.1021/acs. bioconjchem.3c00174
3. Ju, Y., Lee, W. S., Pilkington, E. H., Kelly, H. G., Li, S., Selva, K. J., Wragg, K. M., Subbarao, K., Nguyen, T. H. O., Rowntree, L. C., Allen, L. F., Bond, K., Williamson, D. A., Truong, N. P., Plebanski, M., Kedzierska, K., Mahanty, S., Chung, A. W., Caruso, F., Wheatley, A. K., Juno, J. A., & Kent, S. J. (2022, June 27). Anti-PEG antibodies boosted in humans by SARS-COV-2 lipid nanoparticle mRNA vaccine. ACS Nano, 16(8). https://doi.org/10.1021/acsnano.2c04543
4. Bavli, Y., Chen, B.-M., Gross, G., Hershko, A., Turjeman, K., Roffler, S., & Barenholz, Y. (2023). Anti-PEG antibodies before and after a first dose of Comirnaty® (mRNA-LNP-based SARS-COV-2 vaccine). Journal of Controlled Release, 354, 316-322. https://doi.org/10.1016/j.jconrel.2022.12.039
5. Besin, G., Milton, J., Sabnis, S., Howell, R., Mihai, C., Burke, K., Benenato, K. E., Stanton, M., Smith, P., Senn, J., & Hoge, S. (2019). Accelerated blood clearance of lipid nanoparticles entails a biphasic humoral response of B-1 followed by B-2 lymphocytes to distinct antigenic moieties. Immunohorizons, 3(7), 282-293. https://doi.org/10.4049/immunohorizons.1900029
6. Wang, H., Wang, Y., Yuan, C., Xu, X., Zhou, W., Huang, Y., Lu, H., Zheng, Y., Luo, G., Shang, J., & Sui, M. (2023). Polyethylene glycol (PEG)-associated immune responses triggered by clinically relevant lipid nanoparticles in rats. npj Vaccines, 8, Article 169. https://doi.org/10.1038/s41541-023-00766-z
7. Berger, M., Toussaint, F., Ben Djemaa, S., Laloy, J., Pendeville, H., Evrard, B., Jerôme, C., Lechanteur, A., Mottet, D., Debuigne, A., & Piel, G. (2023). Poly(vinyl pyrrolidone) derivatives as PEG alternatives for stealth, non-toxic and less immunogenic siRNA-containing lipoplex delivery. Journal of Controlled Release, 361, 87-101. https://doi.org/10.1016/j. jconrel.2023.07.031
8. van Zyl, D. G., Mendes, L. P., Semper, R. P., Rueckert, C., & Baumhof, P. (2024). Poly(2-methyl-2-oxazoline) as a polyethylene glycol alternative for lipid nanoparticle formulation. Frontiers in Drug Delivery, 4. https://doi.org/10.3389/fddev.2024.1383038
9. Kang, D. D., Hou, X., Wang, L., Xue, Y., Li, H., Zhong, Y., Wang, S., Deng, B., McComb, D. W., & Dong, Y. (2024). Engineering LNPs with polysarcosine lipids for mRNA delivery. Bioactive materials, 37, 86-93. https://doi.org/10.1016/j.bioactmat.2024.03.017
10. N. Nabar, T. G. Dacoba, G. Covarrubias, D. Romero-Cruz, & P. T. Hammond, Electrostatic adsorption of polyanions onto lipid nanoparticles controls uptake, trafficking, and transfection of RNA and DNA therapies, Proc. Natl. Acad. Sci. U.S.A. 121 (11) e2307809121, https://doi.org/10.1073/pnas.2307809121 (2024).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

### EMBODIMENTS

Embodiment 1. A non-PEGylated nanoparticle comprising:
(a) a lipid nanoparticle (LNP) core comprising an ionizable or cationic lipid and a nucleic acid cargo; and
(b) an outer polyanionic layer wherein:
the LNP core does not comprise a PEG.

Embodiment 2. The nanoparticle of embodiment 1, wherein the nanoparticle does not comprise a nucleic acid-based layer.

Embodiment 3. The nanoparticle of embodiment 1 or 2, wherein the core further comprises a phospholipid.

Embodiment 4. the Nanoparticle of Any One of Embodiments 1-3, Wherein the Core further comprises a sterol.

Embodiment 5. The nanoparticle of any one of embodiments 1-4, wherein the outer polyanionic layer comprises hyaluronic acid (HA), poly-L-glutamate (PLE), poly-L-aspartate (PLD), polyacrylic acid (PAA), dextran sulfate, chondroitin sulfate, fucoidan, heparin sulfate, alginate, pegylated-poly-L-glutamic acid, pegylated-poly-L-aspartic acid, polysialic acid, carboxymethyl cellulose, methacrylate, sulfated polybeta cyclodextrin.

Embodiment 6. The nanoparticle of any one of embodiments 1-5, wherein the outer polyanionic layer comprises PLE.

Embodiment 7. The nanoparticle of any one of embodiments 1-6, wherein the ionizable or cationic lipid is selected from the group consisting of ALC-0315, DLin-MC3-DMA, DLin-KC2-DMA, cKK-E12, C12-200, and SM-102.

Embodiment 8. The nanoparticle of any one of embodiments 1-7, wherein the phospholipid is selected from the group consisting of 1,2-dioleoyl-sn-glycero-3-phosphoethanolamine (DOPE), 1,2-distearoyl-sn-glycero-3-phosphocholine (DSPC), and 1,2-dioleoyl-sn-glycero-3-phosphocholine (DOPC).

Embodiment 9. The nanoparticle of embodiment 8, wherein the phospholipid is 1,2-dioleoyl-sn-glycero-3-phosphoethanolamine (DOPE).

Embodiment 10. The nanoparticle of any one of embodiments 1-8, wherein the sterol is cholesterol.

Embodiment 11. The nanoparticle of any one of embodiments 1-10, wherein the nucleic acid cargo is a polynucleotide.

Embodiment 12. The nanoparticle of embodiment 11, wherein the polynucleotide is selected from the group consisting of mRNA, siRNA, sgRNA, and pDNA.

Embodiment 13. The nanoparticle of any one of embodiments 1-10, further comprising a targeting moiety.

Embodiment 14. The nanoparticle of embodiment 13, wherein the targeting moiety is selected from the group consisting of an antibody, nanobody, affibody, aptamer, cyclodextrin, and a derivative or fragment thereof.

Embodiment 15. The nanoparticle of embodiment 14, wherein the antibody or nanobody is an anti-CD117, anti-CD105, anti-CD90, anti-CXCR4, anti-CD45, anti-CD4, anti-CD8, anti-CD3, anti-CD19, anti-CD20, or anti-ferritin antibody or nanobody.

Embodiment 16. A pharmaceutical composition comprising a plurality of nanoparticles of any one of embodiments 1-15 and a pharmaceutically acceptable excipient.

Embodiment 17. The pharmaceutical composition of embodiment 16, further comprising an additional pharmaceutical agent.

Embodiment 18. A method of delivering a polynucleotide to a subject or cell, the method comprising administering to the subject or contacting the cell with the nanoparticle of any one of embodiments 1-15 or the pharmaceutical composition of embodiment 16 or 17.

Embodiment 19. A method of increasing transfection of a polynucleotide in a cell, the method comprising contacting the cell with the nanoparticle of any one of embodiments 1-15.

Embodiment 20. The method of embodiment 19, wherein the cell is a macrophage.

Embodiment 21. A method of decreasing transfection of a polynucleotide in a cell, the method comprising contacting the cell with the nanoparticle of any one of embodiments 1-15.

Embodiment 22. The method of embodiment 21, wherein the cell is a liver cell.

Embodiment 23. A method of treating or preventing disease in a subject in need thereof, the method comprising administering to the subject the nanoparticle of any one of embodiments 1-15 or the pharmaceutical composition of embodiment 16 or 17.

Embodiment 24. The method of embodiment 23, wherein the disease is a proliferative disease, an immune disorder, genetic disease, or a virus.

Embodiment 25. The method of embodiment 23, wherein the disease is a proliferative disease.

Embodiment 26. A method of stabilizing a non-PEGylated LNP, the method comprising electrostatic layering the non-PEGylated LNP with a polyanion, wherein the nanoparticle does not comprise a nucleic acid-based layer.

Embodiment 27. The method of embodiment 26, wherein the polyanion is selected from the group consisting of hyaluronic acid (HA), polyglutamate (PLE), poly aspartic acid (PLD), and poly acrylic acid (PAA).

1. A non-PEGylated nanoparticle comprising:
(a) a lipid nanoparticle (LNP) core comprising an ionizable or cationic lipid and a nucleic acid cargo; and
(b) an outer polyanionic layer, wherein:
the LNP core does not comprise a poly(ethylene glycol) (PEG) and does not comprise a nucleic acid-based layer.

2. (canceled)

3. The nanoparticle of claim 1, wherein the outer polyanionic layer comprises hyaluronic acid (HA), poly-L-glutamate (PLE), poly-L-aspartate (PLD), polyacrylic acid (PAA), dextran sulfate, chondroitin sulfate, fucoidan, heparin sulfate, alginate, pegylated-poly-L-glutamic acid, pegylated-poly-L-aspartic acid, polysialic acid, carboxymethyl cellulose, methacrylate, sulfated polybeta cyclodextrin.

4. The nanoparticle of claim 3, wherein the outer polyanionic layer comprises PLE or PAA.

5. (canceled)

6. The nanoparticle of claim 1, wherein the ionizable or cationic lipid is selected from the group consisting of ALC-0315, DLin-MC3-DMA, DLin-KC2-DMA, cKK-E12, C12-200, SM-102, C14-4, 306-O12B, and DOTAP.

7. The nanoparticle of claim 6, wherein the ionizable lipid is selected from the group consisting of ALC-0315, cKK-E12, and SM-102.

8. (canceled)

9. The nanoparticle of claim 1, wherein the core further comprises a phospholipid selected from the group consisting of 1,2-dioleoyl-sn-glycero-3-phosphoethanolamine (DOPE), 1,2-distearoyl-sn-glycero-3-phosphocholine (DSPC), hydrogenated soy phosphatidylcholine (HSPC), 1,2-dipalmitoyl-sn-glycero-3-phosphocholine (DPPC), 1,2-distearoyl-sn-glycero-3-phosphoethanolamine (DSPE), 1,2-Dipalmitoyl-sn-glycero-3-phosphoethanolamine (DPPE), 1,2-dioleoyl-sn-glycero-3-phosphocholine (DOPC), 1,2-dioleoyl-sn-glycero-3-phosphoethanolamine-N-[4-(p-maleimidophenyl)butyramide] (MPB-PE), 1,2-dioleoyl-sn-glycero-3-phosphoethanolamine-maleimide (DOPE-Mal), 1,2-dioleoyl-sn-glycero-3-phosphoethanolamine-N-hydroxysuccinimide (DOPE-NHS), 1,2-dioleoyl-sn-glycero-3-phosphoethanolamine-azide (DOPE-azide), 1,2-dioleoyl-sn-glycero-3-phosphoethanolamine-dibenzocyclooctyne (DOPE-DBCO), and 1,2-dioleoyl-sn-glycero-3-phosphoethanolamine-trans-cyclooctene (DOPE-TCO).

10. (canceled)

11. The nanoparticle of claim 1, wherein the core further comprises a sterol selected from the group consisting of campesterol, desmosterol, stigmasterol, lanosterol, cholesterol, and sitosterol.

12-13. (canceled)

14. The nanoparticle of claim 1, wherein the nucleic acid cargo is selected from the group consisting of mRNA, siRNA, sgRNA, and pDNA.

15-16. (canceled)

17. The nanoparticle of claim 1, further comprising at least one targeting moiety or immunostimulatory molecule.

18-19. (canceled)

20. The nanoparticle of claim 17, wherein the at least one targeting moiety or immunostimulatory molecule is an antibody or nanobody selected from the group consisting of an anti-CD117, anti-CD105, anti-CD90, anti-CXCR4, anti-CD45, anti-CD4, anti-CD8, anti-CD3, anti-CD19, anti-CD20, anti-PD1, anti-PDL1, anti-CTLA4, anti-TIM-3, anti-LAG-3, anti-NKG2A, anti-CD73, anti-A2aR, anti-B7-H3, anti-B7-H4, or anti-ferritin antibody or nanobody, or a cytokine selected from the group consisting of interleukin-12 (IL-12), interleukin-2 (IL-2), interferon-γ (IFN-γ), interferon-α (IFN-α), interferon-β (IFN-β), interleukin-15 (IL-15), interleukin-21 (IL-21), interleukin-7 (IL-7), interleukin-15 super agonist (IL-15SA), interleukin-18 (IL-18), tumor necrosis factor alpha (TNF-α), interleukin-10 (IL-10), interleukin-8 (IL-8), TNF-related apoptosis-inducing ligand (TRAIL), FMS-like tyrosine kinase 3 ligand (FLT3LG), and variants thereof.

21-28. (canceled)

29. A pharmaceutical composition comprising a plurality of nanoparticles of claim 1 and a pharmaceutically acceptable excipient.

30. (canceled)

31. A method of delivering a polynucleotide to a subject or cell, the method comprising administering to the subject or contacting the cell with the nanoparticle of claim 1.

32. The method of claim 31, further comprising reducing liver transfection relative to an unlayered nanoparticle.

33. (canceled)

34. The method of claim 31, wherein the particle is administered via intravenous injection, intraperitoneal injection, intratumoral injection, intramuscular injection, intradermal injection, topically, trans-dermally, oral intake, inhalation, intrathecally, intranasally, buccal, intravesically, intra-arterially, or through forced methods.

35. A method of modulating transfection of a polynucleotide in a cell, the method comprising contacting the cell with the nanoparticle of claim 1.

36-44. (canceled)

45. A method of treating or preventing disease in a subject in need thereof, the method comprising administering to the subject the nanoparticle of claim 1.

46. The method of claim 45, wherein the disease is a proliferative disease, an immune disorder, genetic disease, or a virus.

47. The method of claim 46, wherein the proliferative disease is ovarian cancer.

48. A method of stabilizing a non-PEGylated LNP, the method comprising electrostatic layering the non-PEGylated LNP with a polyanion, wherein the nanoparticle does not comprise a nucleic acid-based layer.

49-51. (canceled)

52. A kit comprising:
a nanoparticle of claim 1; and
instructions for using the particle.
